(19) United States
(12) Patent Application Publication
(10) Pub. No.: US 2016/0338858 A1
(43) Pub. Date: Nov. 24, 2016

(54) PROSTHETIC SOCKET LINER GARMENT

(71) Applicant: LIM Innovations, Inc., San Francisco, CA (US)

(72) Inventors: Garrett Ray Hurley, San Francisco, CA (US); Juan Jacobo Cespedes, San Francisco, CA (US); Jesse Robert Williams, San Francisco, CA (US); Ryan M. Satcher, San Francisco, CA (US)

(21) Appl. No.: 15/157,894

(22) Filed: May 18, 2016

Related U.S. Application Data
(60) Provisional application No. 62/163,577, filed on May 19, 2015.
(60) Provisional application No. 62/237,232, filed on Oct. 5, 2015.
(60) Provisional application No. 62/259,855, filed on Nov. 25, 2015.

(51) Int. Cl. A61F 2/78

(52) U.S. Cl. CPC A61F 2/7812; A61F 2002/7818; A61F 2002/785; A61F 2210/0071; A61F 2220/005; A61F 2230/0017

(57) ABSTRACT

A liner garment for a prosthetic socket configured to accommodate a residual limb of a patient may include a main body, a gel cup and at least one support material layer. The main body may include an elastic, fluid permeable material layer extending around a full circumference of the liner garment and having a closed distal end and an open proximal end for accepting the residual limb of the patient. The gel cup is fluid impermeable and is positioned on the inner surface of the main body at its distal end. The support material layer is located on a portion of the outer surface of the main body and is made of a material that is less elastic than the fluid permeable material layer of the main body.

### CROSS-REFERENCE TO RELATED PATENT APPLICATIONS

The present application claims priority to U.S. Provisional Patent Application No. 62/163,577, filed May 19, 2015, entitled “An Integrated Multi-Material Prosthetic Socket Liner Garment;” 62/237,232, filed Oct. 5, 2015, entitled “An Integrated Multi-Material Prosthetic Socket Liner Garment;” and 62/259,855, filed Nov. 25, 2015, entitled “An Integrated Multi-Material Prosthetic Socket Liner Garment.” The entireties of each application above are herein incorporated by reference.

### INCORPORATION BY REFERENCE

All publications and patent applications referenced in this specification, including the above-listed patent applications, are hereby incorporated fully by reference herein, to the same extent as if each such individual publication or patent application were specifically and individually indicated to be so incorporated by reference.

### TECHNICAL FIELD

The present technology relates to the field of prosthetic and orthotic devices. More particularly, the technology relates to a prosthetic socket liner garment.

### BACKGROUND

Polymeric prosthetic socket liners, commonly referred to as gel liners, are widely used as an interface between the surface of a residual limb and a prosthetic socket that grasps the residual limb and provides continuity of function between the residual limb and more distal prosthetic components. Prosthetic socket liners provide padding or pressure distribution surfaces that create greater comfort for the patient and also participate in suspension of the prosthetic socket on the residual limb.

“Comfort,” in the context of prosthetic socket liners, is a serious clinical consideration. If comfort is anything less than fully satisfactory, functionality of the prosthetic device (and thus long term compliance) will very likely be unsatisfactory. “Suspension,” in this context, refers to maintenance of the prosthetic socket on the residual limb—in other words, suspension refers to the prosthetic socket not slipping off the limb, not pistoning up and down on the limb, and not rotating around the limb. Suspension of a prosthesis typically relies both on the tenacity with which a socket liner maintains its grasp on the residual limb and also on the tenacity with which the socket maintains its grasp on the liner. Satisfactory performance of the prosthesis as a whole depends on many factors, but the quality of the fit of the socket on the residual limb, an optimal liner solution, and the overall quality of suspension of the socket are important.

Polymeric prosthetic liners typically include materials such as silicone, urethane, and thermoplastic elastomer gels in the form of blends and block copolymers, as well mineral oils. Such liners may be referred to generically herein as “gel liners.” Gel liners or liner garments are conformable and, when correctly sized, are donned and removed with relative ease. Liners fit closely against the skin and may further be subjected to an internal evacuating pressure that ensures a close fit. If the fit creates an effective seal, evacuation may create a subatmospheric pressure in the interfacing space between the surface of the residual limb and the liner. These features broadly contribute to the liner's role in suspension of the prosthetic socket, but they also create a closed environment that supports the build-up of body-generated moisture and heat around the residual limb. Excess heat and moisture are detrimental to the health of the residual limb. These conditions are damaging to skin integrity and create conditions that support bacterial colonization.

Various approaches to removing accumulated moisture are known, including passive distal drainage of the liner and assisted drainage by way of pumps. Nevertheless, socket liner solutions that provide effective suspension are still complicated by accumulation of moisture and heat. Therefore, it would be desirable to have improved prosthetic socket liner technology solutions that preserve or enhance the suspension of a prosthetic device on a residual limb, and which alleviate at least some of the moisture and heat driven consequences of conventional liner solutions.

### SUMMARY

In one aspect, embodiments of the invention are directed to a liner garment for a prosthetic socket that is configured to accommodate a residual limb of a patient, and include two distinct portions, a distal garment portion and a proximal sleeve portion. The distal garment portion of these embodiments is configured to accommodate a distal end of the residual limb, the distal garment portion comprising a fully encompassing internal gel layer that is substantially fluid-impermeable. The proximal sleeve portion of these embodiments includes an elastic fabric configured to accommodate at least a portion of the residual limb, an internal surface and an external surface, and the elastic fabric is fluid-permeable. The liner garment, as a whole, has an internal surface and an external surface.

In some embodiments of the liner garment, the distal portion and the proximal liner sleeve portion form an integral garment, with portions of the garment integrated by way of any suitable type of bonding, annealing, gluing and/or interweaving. In some embodiments, the garment includes any of multiple materials and/or multiple layers, which are integrated into a single garment by way of bonding, gluing, and/or interweaving. In some embodiments of the liner garment, the proximal sleeve portion and the distal garment portion are adapted to provide distinct internal compartments within an interior of the liner garment for a residual limb hosted therein. In some embodiments, a proximal may be breathable, and a distal compartment may be substantially hermetically sealed.

In various embodiments of the liner garments, the inner gel layer of the distal portion of the liner garment may include any suitable material, such as silicone, urethane, and thermoplastic elastomer gels, in the form of blends and block copolymers. In some embodiments, the fully encompassing inner gel layer of the liner garment is restricted to the distal portion of the garment. In particular embodiments, the fully encompassing inner gel layer of the liner garment comprises a length in the range of about 100 mm to about 170 mm. In some of the liner garment embodiments, the elastic fabric of the proximal liner sleeve portion includes a polyester-polyurethane copolymer fiber (i.e., spandex).

In typical liner garment embodiments, the proximal liner sleeve portion of the liner has a tubular configuration with a vertical axis and a circumferential aspect orthogonal to the vertical axis, and the proximal liner sleeve portion has a woven fabric with a biased stretchability, such that stretch is allowed circumferentially but is substantially constrained or disallowed vertically. In some liner garment embodiments, the proximal liner sleeve portion has a pattern of polyurethane laminated on the external surface of the elastic fabric or integrated into the elastic fabric, such that it is exposed on the external surface. In some embodiments, the proximal liner sleeve portion includes a non-stretch webbing, applied to the internal surface or integrated into the internal surface of the fabric. Such webbing may be in the form of strips or patterns that include open spaces. In some embodiments of the liner garment, the proximal liner sleeve portion includes a non-stretch webbing applied to the external surface or integrated into the external surface of the fabric. In some liner garment embodiments, the proximal liner sleeve portion includes tensioning straps arranged on the external surface.

In some embodiments of the liner garment, the distal end of the liner may include an umbrella disposed on an external surface of the liner garment. Embodiments of the umbrella are generally saucer shaped and have a concave proximal surface that conforms to a convex aspect of the distal end of the garment liner. In some embodiments of the liner garment, the umbrella has a pentagonal periphery. The five points of the pentagonal shape may be arranged to be disposed over five centrally converging seams disposed at the distal end of the liner garment, the five seams forming a joining of the edges of darts in a flat pattern from which the liner garment is fabricated. In some liner garment embodiments, the umbrella has a distal surface, and a set of radiating ribs is disposed on the distal surface. This set of radiating ribs on the distal surface of the umbrella is configured to engage a complementary set of teeth and intervening slots disposed on the proximal surface of a distal funnel disposed at the distal-most aspect of a cavity of a prosthetic socket. In some umbrella embodiments, the surface area of the umbrella (and, accordingly, the surface area in contact with the liner garment embodiments) ranges between about 15.9 cm² and about 29.2 cm². In related embodiments, the surface area of the umbrella may be greater than any of 15 cm², greater than 20 cm², greater than 25 cm², or greater than 30 cm².

In some embodiments of the liner garment, the prosthetic socket includes a distal funnel disposed within a distal-most site within a central, proximal facing cavity of the prosthetic socket. In some embodiments, the distal funnel has a proximal surface with a set of teeth and intervening slots configured to complement a shape of radiating ribs disposed on a distal surface of an umbrella disposed at the distal end of the liner garment.

Some embodiments of the liner garment have a cable-based tensioning mechanism. For example, the cable based tensioning mechanism may be disposed on an external surface of the liner garment, and arranged in multiple circumferential loops there around. In some of these particular embodiments, the cable of the tensioning mechanism is enclosed in a plastic sheath, which is adhered to the external surface of the liner by one or more layers of thermoplastic. In a second example, the cable-based tensioning mechanism may be disposed within a fabric layer of the liner garment, arranged in multiple circumferential loops.

In typical embodiments of the liner garment, the distal end of the garment has a distal connection feature configured to attach to a distal internal site within a prosthetic socket. Such distal connection feature may include an umbrella formed of thermoplastic and adherent to the distal end of liner garment, the umbrella having a distally directed feature configured to engage the distal and internal aspect of a prosthetic socket.

In another aspect, a prosthetic socket configured to accommodate a residual limb of a patient may include a prosthetic socket frame, a prosthetic socket liner garment, an umbrella bonded to a distal aspect of the liner garment, and a distal funnel. The distal funnel may be secured within the prosthetic socket frame, The umbrella and the distal funnel may be locked together in a non-rotatable manner relative to each other, thereby securing the prosthetic socket liner garment within the prosthetic socket frame in a manner that precludes rotation of the liner garment with respect to the prosthetic socket frame.

In yet another aspect of the invention, a liner garment system for a prosthetic socket is provided that is configured to accommodate a residual limb of a patient. Embodiments of the liner garment system include a main body formed by an elastic, fluid permeable material layer extending around a full circumference of the liner garment and having an inner surface, an outer surface, a closed distal end and an open proximal end for accepting the residual limb of the patient. Embodiments further include a fluid impermeable gel cup positioned on the inner surface of the main body at its distal end, wherein the gel cup is configured to accommodate a distal end of the residual limb, and the gel cup does not extend to the proximal end of the main body. Embodiments may further include at least one support material layer on a portion of the outer surface of the main body, wherein the at least one support material layer includes a material that is less elastic than the fluid permeable material layer of the main body. Embodiments further include a supportive umbrella attached to the outer surface of the distal end of the main body. And embodiments still further include a distal funnel configured for placement at a distal-most end of a cavity of the prosthetic socket, wherein the distal funnel has a proximal surface that is complementary in shape to a distal surface of the umbrella.

In some embodiments, a prosthetic liner garment may be included with a prosthetic socket device that also has a thermoplastic distal support cup disposed within the internal and distal aspect of a prosthetic socket. In particular embodiments, the distal support cup may be formed of an ethylvinylacetate-polycaprolactone (EVA-PCL) copolymer that has a thermolabile temperature low enough that it can be molded directly to the distal end of a residual limb (albeit with an optional intervening heat protective fabric) to custom mold the distal cup for the patient. In some embodiments, the distal cup and the liner garment may be bonded together. Aspects of this technology are described in U.S. patent application Ser. No. 14/659,433 of Hurley et al., as filed on Mar. 19, 2015, and in U.S. patent application Ser. No. 14/951,878 of Hurley et al., as filed on Nov. 25, 2015.

These and other aspects and embodiments are described in greater detail below, in reference to the attached drawing figures.

### BRIEF DESCRIPTION OF THE FIGURES

FIG. 1 is a side perspective view of a liner garment, showing proximal and distal portions of the garment, as well as an internal gel layer restricted to the distal portion, according to one embodiment;

FIG. 2A is a top perspective view of an embodiment of a prosthetic socket liner garment, showing various layers internal and external to a substantially contiguous neoprene fabric-foam-fabric layer;

FIG. 2B is a top perspective view (steeper than that of FIG. 2A) of an embodiment of a prosthetic socket liner garment, showing an interior aspect of the garment and a gel cup disposed and bonded within the distal interior cavity;

FIG. 2C is a bottom perspective view of an embodiment of a prosthetic socket liner garment, showing an exterior aspect of the garment and an exterior pentagonal umbrella bonded at the distal end;

FIG. 3A is an exploded half-pipe view of an embodiment of a prosthetic socket liner garment, showing various layers internal and external to a substantially contiguous neoprene fabric-foam-fabric layer, an internally disposed gel cup, and an externally disposed umbrella;

FIG. 3B is an exploded view of a flat pattern of an embodiment of a prosthetic socket liner garment, showing various layers internal and external to a substantially contiguous neoprene fabric-foam-fabric layer, an internally disposed gel cup, and an externally disposed umbrella;

FIG. 3C is an exploded view of a neoprene fabric-foam-fabric layer, according to one embodiment;

FIG. 4A is an external view of a gel cup portion of a prosthetic liner, showing fluid movement channels disposed on the internal surface, according to one embodiment;

FIG. 4B is an external view of a gel cup portion of a prosthetic liner, showing fluid movement channels disposed on the internal surface, according to an alternative embodiment;

FIG. 4C is a cross-sectional detail view of a fluid movement channels, as in FIGS. 4A-4B;

FIG. 5 is a schematic view of an exemplary step in the fabrication of an embodiment of a prosthetic socket liner garment in which a gel cup, supported on a mandrel, is being inserted into an awaiting liner garment shell;

FIGS. 6A-6C are modular sizing options for an embodiment of a prosthetic socket liner garment. FIG. 6A is a small size; FIG. 6B is a medium size; and FIG. 6C is a large size;

FIG. 7 is a schematic rendering of a flat pattern of a prosthetic socket liner garment, showing regions of substantial inelasticity and regions of elastic availability and breathability, according to one embodiment;

FIG. 8A is a side perspective view of an embodiment of a prosthetic socket liner garment with a tensioning cable system applied circumferentially around the external surface of the garment;

FIG. 8B is a detailed cross-sectional view of a cable within a sheath that is bonded to the external surface of the prosthetic socket liner of FIG. 8A;

FIG. 9A is a side perspective view of a cable tensioning system disposed within a woven fabric portion of prosthetic liner garment, in which the cables are not visible on the external surface of the liner garment, according to one embodiment;

FIG. 9B is a view of the garment of FIG. 9A, with an external woven layer removed, providing a view of the cable tensioning system;

FIG. 9C is a detailed, cross-sectional view of cables disposed within liner fabric, from FIG. 9B;

FIGS. 10A and 10B are perspective views of a prosthetic socket liner garment and a brace portion of the garment, respectively, with a bracing pattern integrated into the fabric, the brace having regions of reduced elasticity compared to the background matrix of the fabric and also having tensioning regions;

FIGS. 11A-11P are various views of an embodiment of a pentagonal umbrella and a distal rotational locking funnel for a prosthetic socket liner garment, according to one embodiment;

FIGS. 11A and 11B are top perspective and bottom perspective views, respectively, of the pentagonal umbrella;

FIG. 11C is a top perspective view of the distal rotational locking funnel;

FIGS. 11D-11F are top perspective, side, and bottom perspective views, respectively, of the umbrella supported by the distal rotational locking funnel;

FIG. 11G is a top perspective view of the umbrella and distal rotational locking funnel, showing a reference plane that corresponds to the reference line in FIG. 11H;

FIG. 11H is a top view of the umbrella, with a cross-sectional reference line pertinent to FIGS. 11I and 11J;

FIG. 11I is a detailed side view of the engagement of distal ribs of the umbrella and proximal teeth and spaces of the distal rotational locking funnel;

FIG. 11J is a side face view of the umbrella above the distal rotational locking funnel, with a reference line that corresponds to a cross-sectional slice, as seen in FIG. 11K;

FIG. 11K is a cross-sectional view of the umbrella above the distal rotational locking funnel, the cross-section corresponding to the reference line seen in FIG. 11J; and

FIGS. 11L-11P are side, cross-sectional, perspective, exploded and bottom views, respectively, of a prosthetic socket liner garment, with the pentagonal umbrella bonded to its distal face, according to one embodiment.

### DETAILED DESCRIPTION

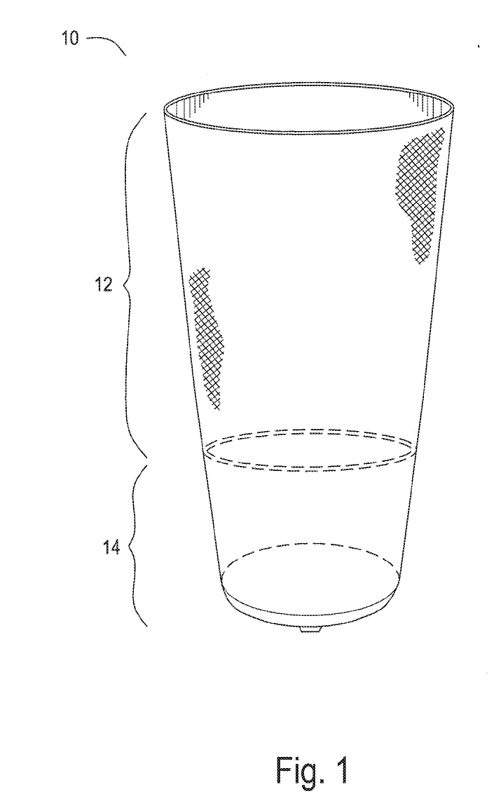

Referring to FIG. 1, in one embodiment, a liner garment 10 for a prosthetic socket (or “prosthetic socket liner”) may have a proximal portion 12 and a distal portion 14. An internal gel layer (not shown) is restricted to distal portion 14. Proximal portion 12 forms a breathable compartment, whereas, by virtue of the internal gel layer, distal portion 14 forms a substantially sealed compartment, disallowing passage or air or fluid. The composition of the wall of proximal portion 12, in addition to being breathable, also functions as a wicking substrate that encourages movement of fluid away from the surface of a residual limb and onto the high surface area of the wicking substrate, wherefrom it eventually evaporates into the external environment.

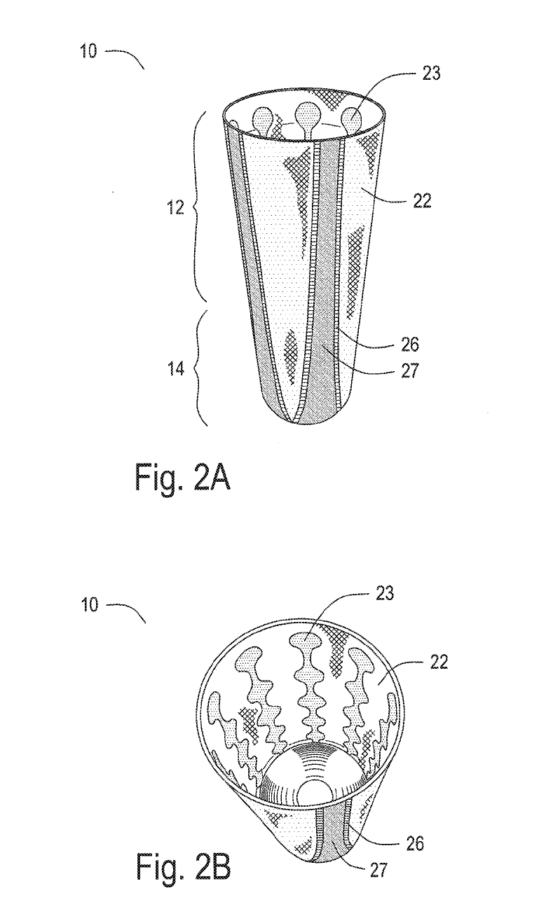

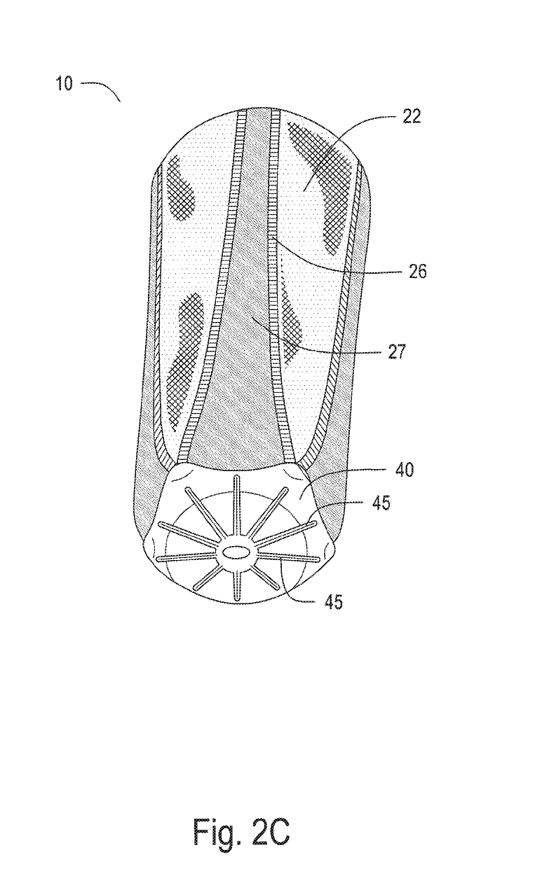

FIGS. 2A-2C are various views of prosthetic socket liner 10, building out detail of the basic structure shown in FIG. 1, with breathable proximal portion 12 and hermetically sealed distal portion 14. FIG. 2A shows various layers internal and external to a substantially contiguous neoprene fabric-foam-fabric layer 22. Neoprene is but one example of a padded, breathable composition—alternative embodiments of prosthetic socket liner 10 may include other materials.

FIG. 2B is a top perspective view (steeper than that of FIG. 2A) prosthetic socket liner 10, showing an interior aspect of the garment and a gel cup disposed and bonded within the distal interior cavity. FIG. 2C shows a bottom perspective view of prosthetic socket liner garment 10, showing an exterior aspect of the garment and an exterior pentagonal umbrella 40 bonded at the distal end.

On the internal surface of neoprene-based breathable layer 22, an arrangement of an insular or circumferentially discontinuous silicone gel applique 23 is disposed, typically applied by a silkscreen method. These silicone gel islands function to enhance the adherence of the liner garment 10 on the residual limb, while (by not being circumferentially discontinuous) they preserve the breathable aspect of proximal portion 12. The pattern of insular gel patterns on the internal surface of liner garment 10 may assume any suitable configuration.

As seen in FIGS. 2A-2B, in particular, a discontinuous structural layer or set of features is adhered to the external side of neoprene-based breathable layer 22. In general, these structural or support features are less elastic than neoprene-based breathable layer 22. In this particular example, a semi-elastic layer 26 is adhered to the external surface of layer 22, and a substantially inelastic layer 27 is adhered to the external surface, and within the surface area boundaries, of semi-elastic layer 26.

As seen in FIG. 2C, a hard plastic umbrella 40 is disposed at the distal end of prosthetic socket liner garment 10. In this bottom view, radiating ribs 45 are shown. These distally projecting features are configured to engage a complementary set of proximally projecting features on a distal funnel 60 (described further below). This arrangement of engaging features comprises an anti-rotational mechanism that rotationally stabilizes prosthetic socket liner 10 within a prosthetic socket frame.

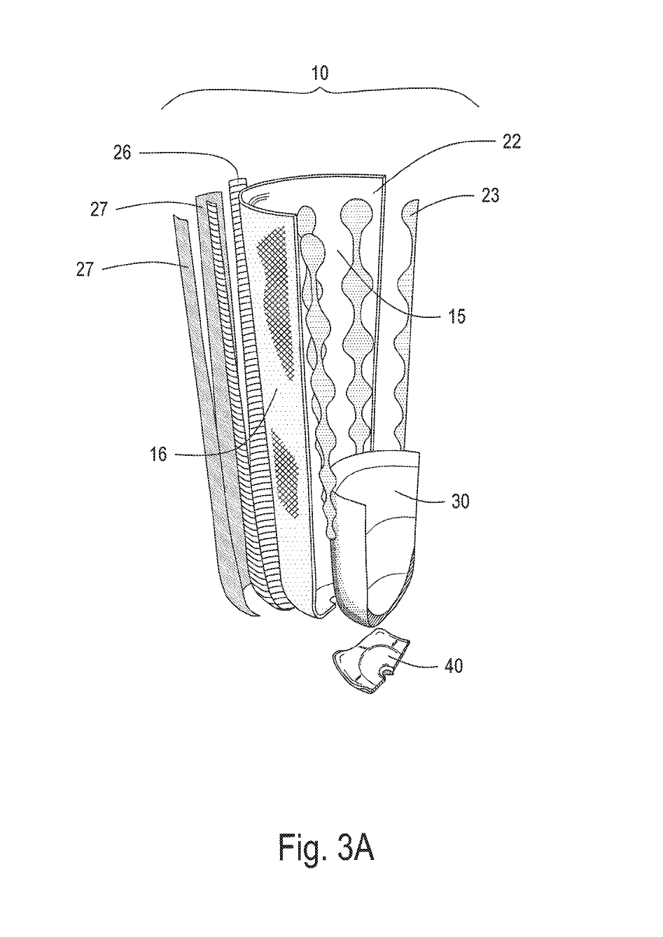

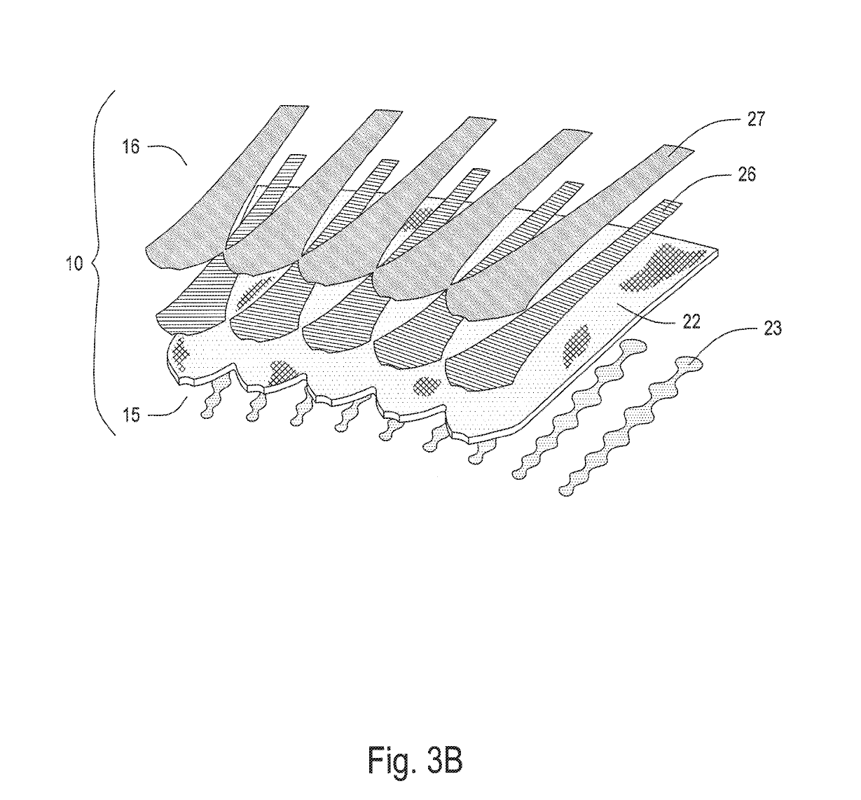

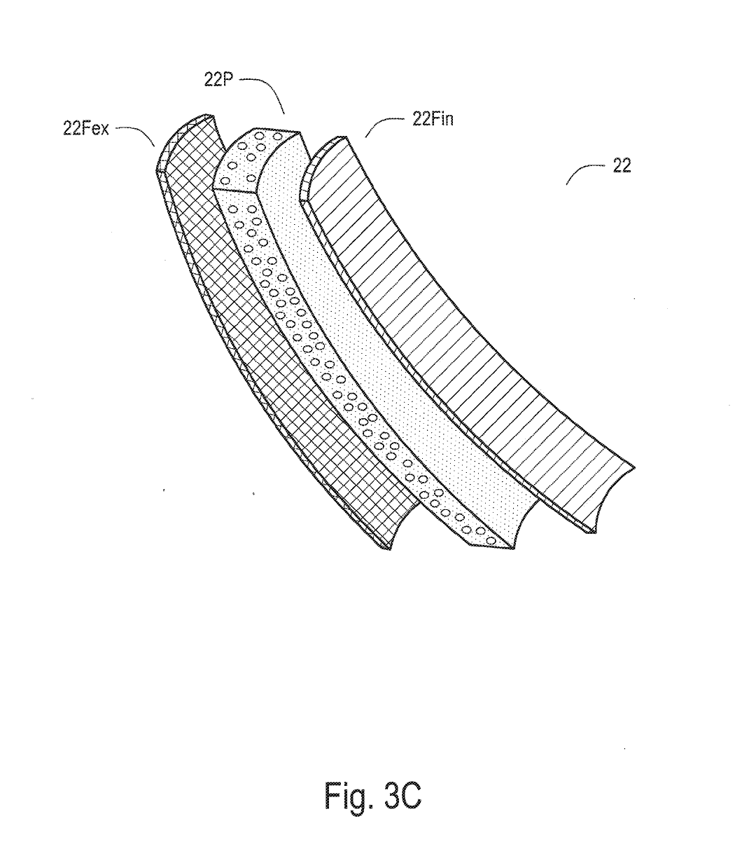

FIGS. 3A-3C show various exploded and cross-sectional views of the wall of prosthetic socket liner garment 10, the wall having an internal surface 15 and an external surface 16. FIG. 3A provides an exploded half-pipe view of prosthetic socket liner garment 10, showing various layers internal and external to a substantially contiguous neoprene fabric-foam-fabric breathable layer 22, an internally disposed gel cup 30, and an externally disposed umbrella 40. As noted above, a semi-elastic strip or layer 26 is disposed external to foam-fabric-foam breathable layer 22, and a substantially inelastic strip or layer 27 is disposed external to semi-elastic layer 26. A pattern of gel 23, arranged so that there is no circumferential continuity, is disposed internal to the internal surface of foam-fabric-foam breathable layer 22.

FIG. 3B shows an exploded view of a flat pattern of prosthetic socket garment 10, showing various layers internal and external to a substantially contiguous neoprene fabric-foam-fabric layer 22. A layer of intermittent or insular (i.e., not contiguous or circumferential) silicone gel layer 23 is disposed on internal surface 15 of breathable layer 22. External to breathable layer 22 are adherent semi-elastic pieces 26, and adhering to them are inelastic pieces 27.

FIG. 3C shows an exploded view of a neoprene-based fabric-foam-fabric breathable layer 22. A neoprene padding layer 22P is sandwiched between an external fabric 22Fex and an internal fabric 22Fin.

In one example, semi-elastic layer 26 is provided by Lamblon™, a product of Daewon Plastic Division (Korea and Vietnam), and includes 77% polyester and 23% polyurethane film, described as polyurethane synthetic suede with microfiber knit backer. In one example, substantially inelastic layer 27 is provided by Thalia™, also a product of Daewon Plastic Division, which includes 30% polyurethane coating and 70% TPU, described as “Premium polyurethane hot-melt film”.

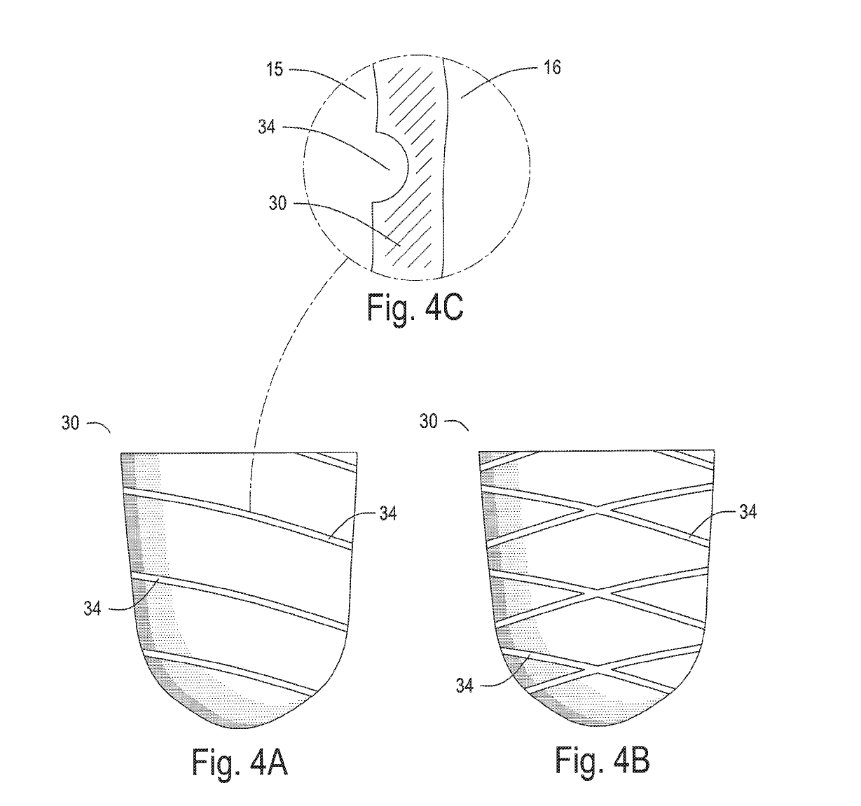

FIGS. 4A-4C show embodiments of a system of fluid movement channels 34 disposed on the internal surface of gel cup 30. Channels 34 have an open half-pipe configuration on internal surface 15 of gel cup 30 that allows capture of perspired fluid from the surface of a hosted residual limb. Once fluid is within the channel 34, there is net movement proximally, as fluid can escape when it encounters fabric-foam-fabric layer 22 by wicking, and ultimately, by evaporating from layer 22 into the external environment.

FIG. 4A shows an external view of a gel cup portion 30 of a prosthetic garment embodiment 10, showing, in particular wicking channels 14 disposed on the internal surface 15 thereof. In this embodiment, channels run parallel to each other, with a pitch that takes the channels in a proximal direct. FIG. 4B shows a variation of the pattern seen in FIG. 4A; channels 34 are arranged as two groups within which channels are parallel to each other, but the groups have oppositely directed pitches, thus forming an intersecting pattern. FIG. 4C is a cross-sectional detail view of channels 34, showing their internally open aspect.

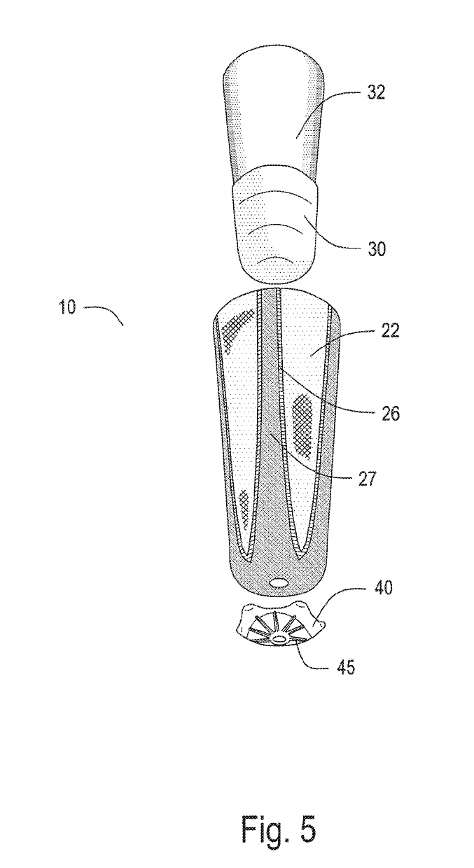

FIG. 5 shows a schematic view of a step in the fabrication of prosthetic socket liner garment 10, according to one embodiment, in which a gel cup 30, supported on a mandrel 32, is being inserted into an awaiting liner garment shell (or foam-fabric-foam layer 22). A layer of bonding adhesive is layered over the distal end of gel cup 30, prior to being inserted into awaiting prosthetic liner garment shell, and once inserted, gel cup 30 and liner garment 10 are bonded together.

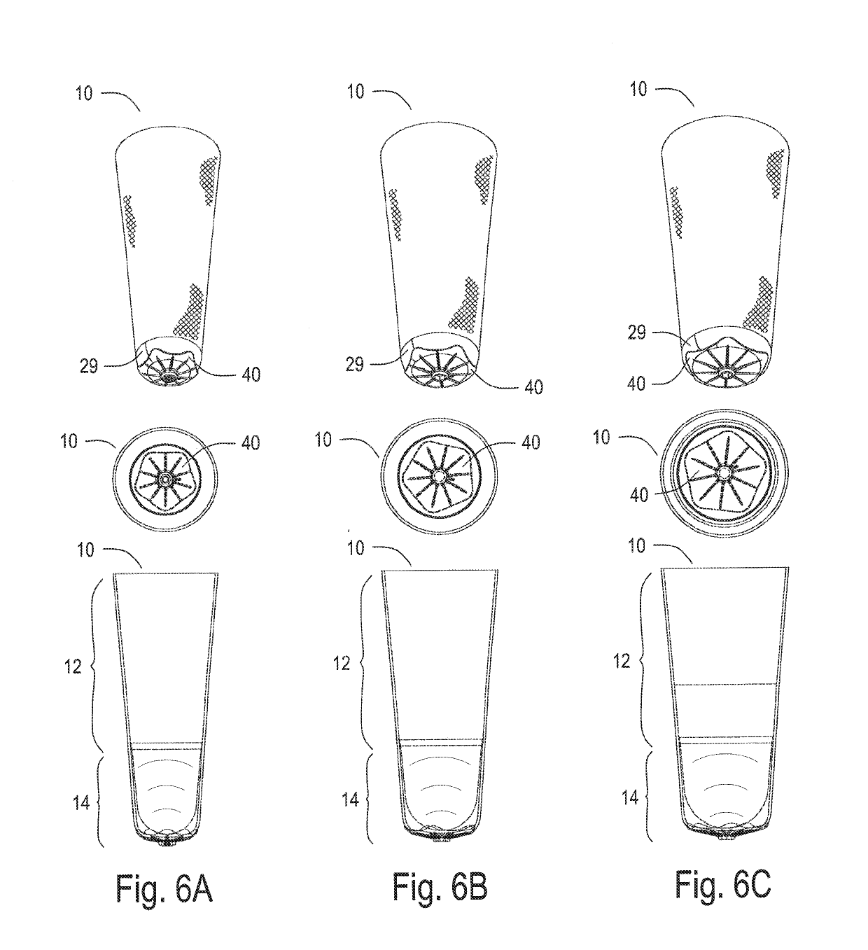

FIGS. 6A-6C show modular sizing options for prosthetic socket liner garment 10, according to one embodiment. FIGS. 6A-6C, in sequence, show small, medium, and large size variations of the garment. The top portion of each of FIGS. 6A-6C shows an upwardly directed perspective view of a differently sized liner garment 10, a bottom face view, and a side view. Different sizing options, which may be found in an inventory of prosthetic liner garments 10, enable a modular approach to fitting amputee patients and are consistent with options associated with sizing and modular assembly of prosthetic socket frame embodiments.

As noted elsewhere, a relatively large surface area of contact between an umbrella 40 and the main body of a liner garment 10 is advantageous for maintaining stability of the liner garment on the residual limb. Typical embodiments for umbrella 40 have a diameter in the range of about 4.5 cm to about 6.1 cm. Accordingly, the surface area interface between umbrella 40 and liner garment 10, in various embodiments ranging between small and large sizes, is in the range of between about 15.9 cm² and about 29.2 cm². Accordingly, in some embodiments, the surface area of umbrella 40 is at least 15 cm²; in some embodiments it is at least 20 cm²; in some embodiments it is at least 25 cm²; and in some embodiments it is at least 30 cm². In more general terms, umbrella 40 usually covers and embraces the substantial entirety of the distal rounded end of liner garment 10.

FIG. 7 shows a schematic rendering of a flat pattern of an embodiment of a prosthetic socket liner garment 10FP, showing several substantially inelastic regions 25 and breathable layer 22. Proximal portion 12 is shown in the top portion of the pattern, and distal portion 14 is shown in the bottom portion of the pattern. As shown in FIGS. 2A-3C, breathable layer 22 is substantially contiguous throughout liner garment 10, including an underlying presence in inelastic regions 25. In some embodiments, structural inelastic regions 25 (or “patterns”) may include an inner, semi-elastic layer 23 (FIGS. 2A, 2B), which supports an inelastic layer external to it. Inelastic structural regions 25 may be arranged in a biomechanically appropriate manner, providing regions of compression or heightened pressure at particular regions of a hosted residual limb that benefit from such support. Areas of a residual limb that directly contact breathable layer 22 receive a lower level of pressure, compared to areas of the limb that line up against inelastic, structural regions 25. In some embodiments of methods of fabricating prosthetic socket liner garments 10, the pattern by which layer 25 is distributed may be customized to the particular anatomy of an individual patient.

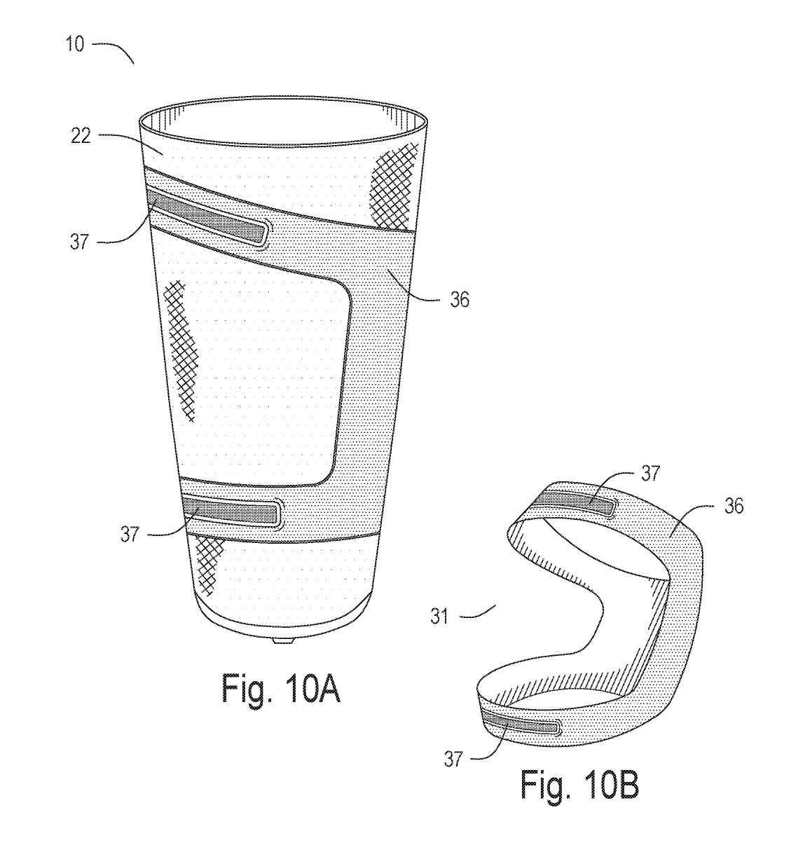

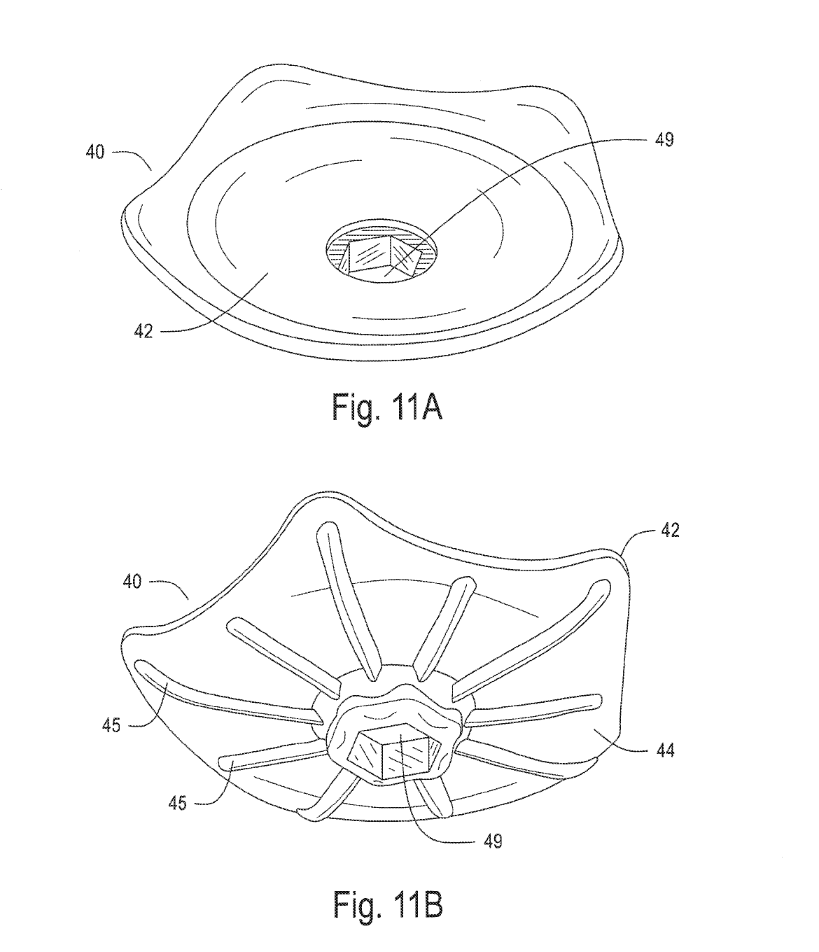

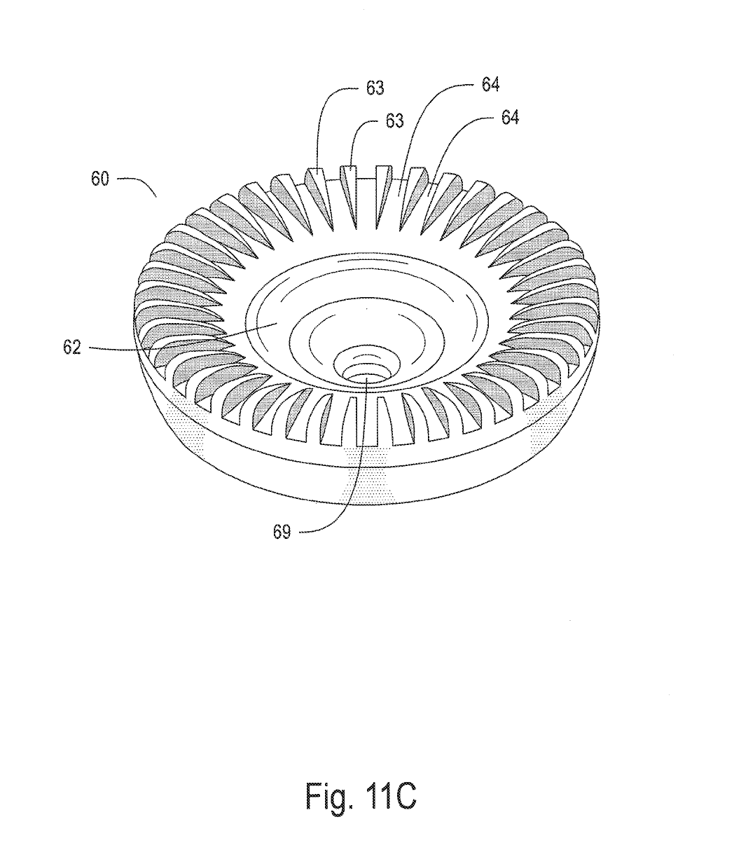

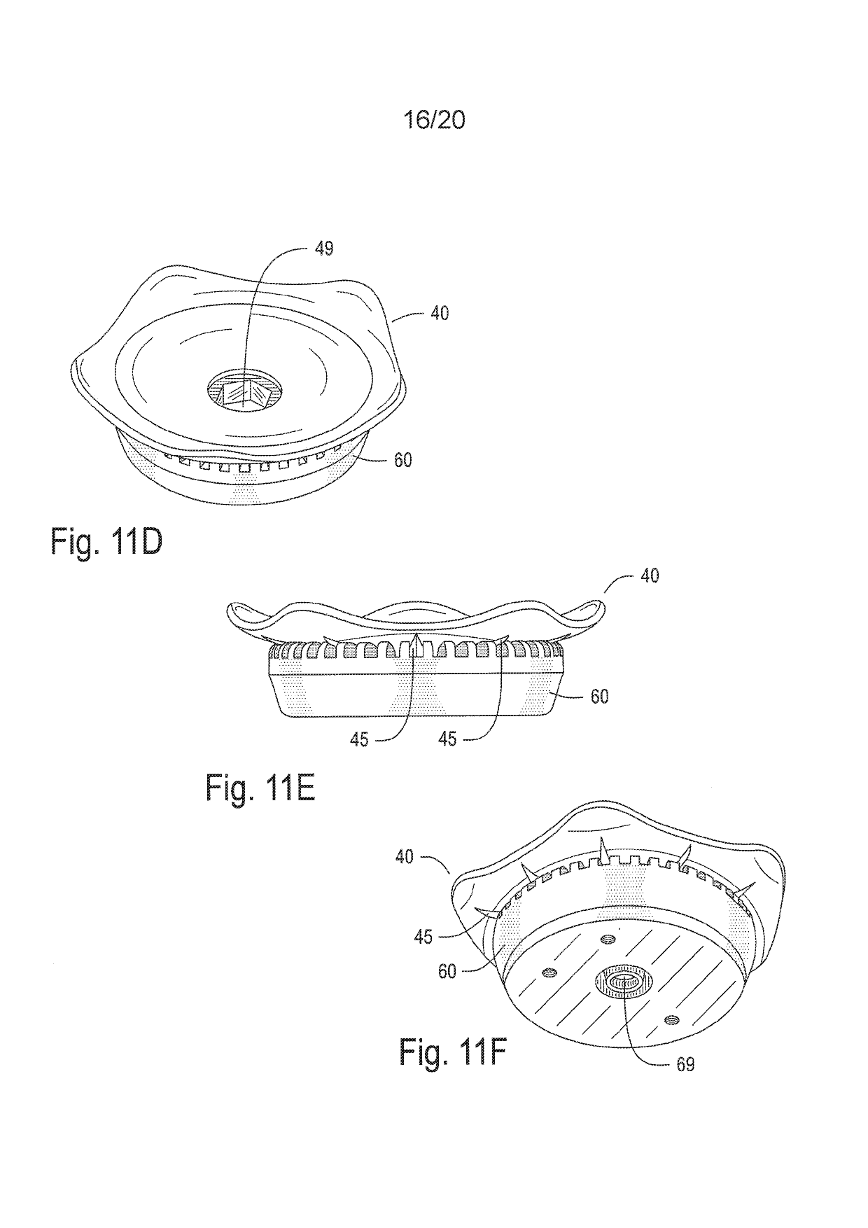

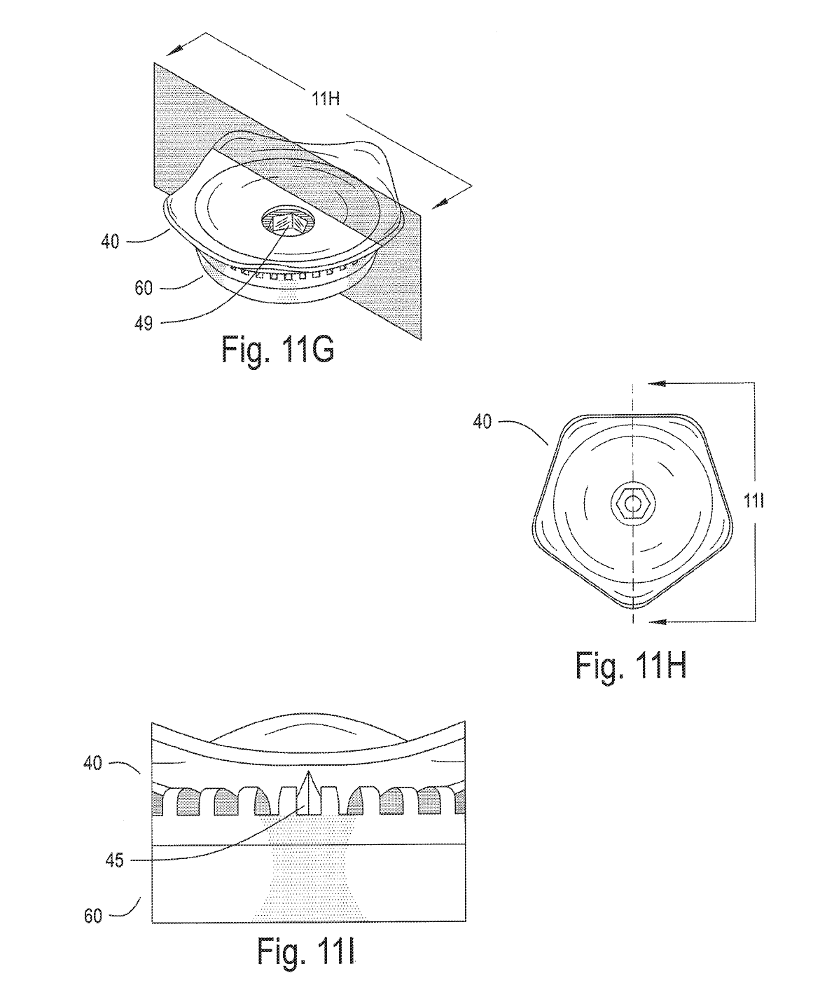

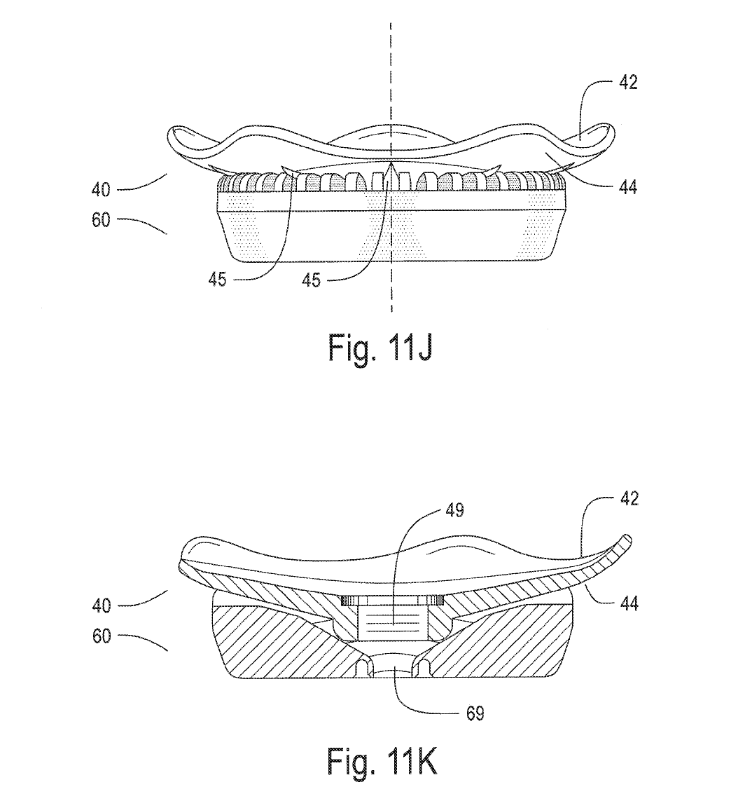

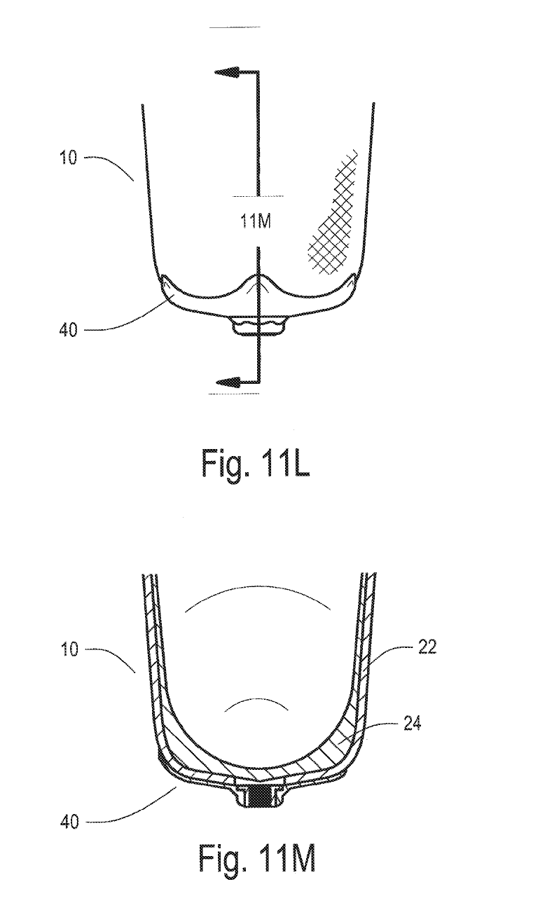

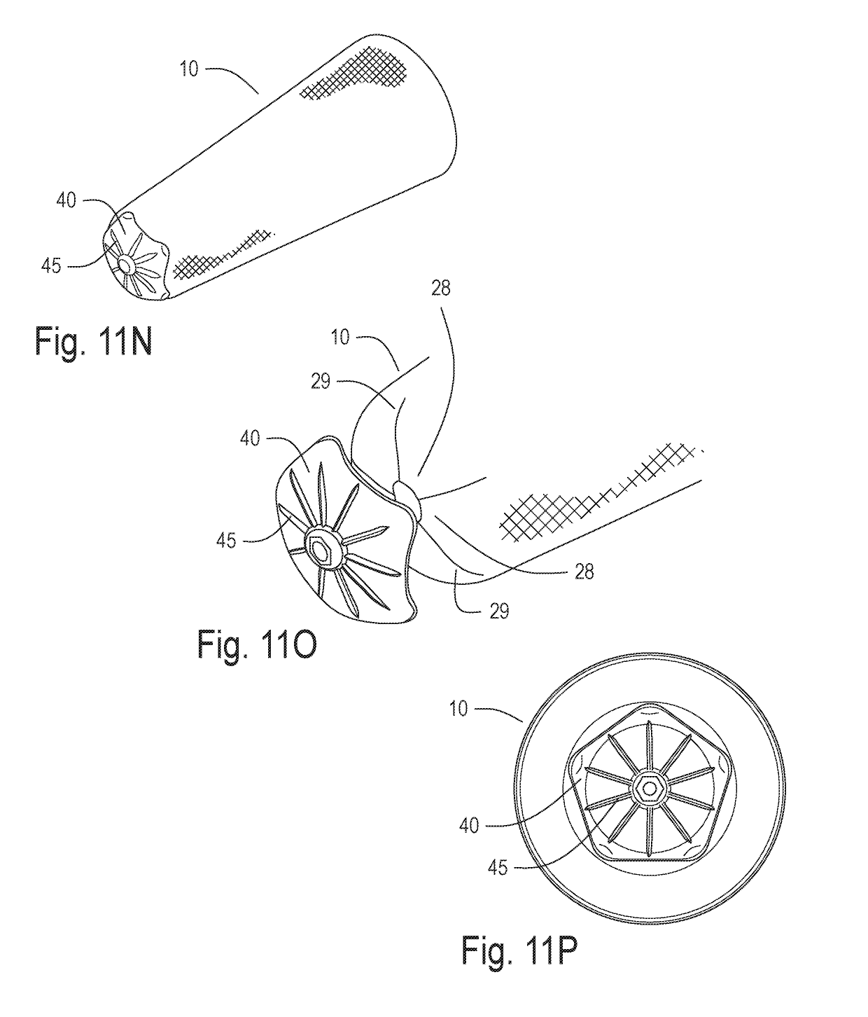

To configure flat pattern 10FP into a cylindrical liner garment 10, the left and right vertical sides flat pattern 10FP may be bonded together. Labels arrayed across both the top and the bottom of flat pattern 10FP indicate the orientation of circumferential regions (lateral L, posterior P, medial M, and anterior A), as they align on the residual limb when prosthetic socket liner garment 10 is being worn. Dart points or peaks 28 are disposed along the distal edge of liner garment 10. As a step in configuring a liner garment 10 into a cylindrical form, dart peaks 28 may be brought together and bonded to create dart seams 29, as shown in FIG. 11O.

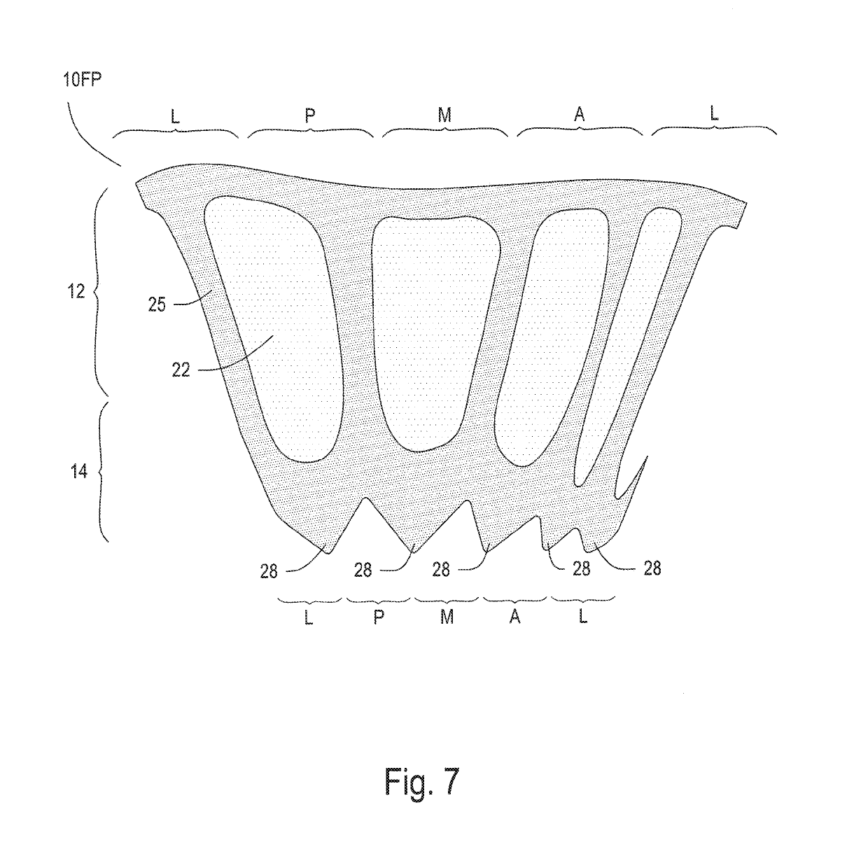

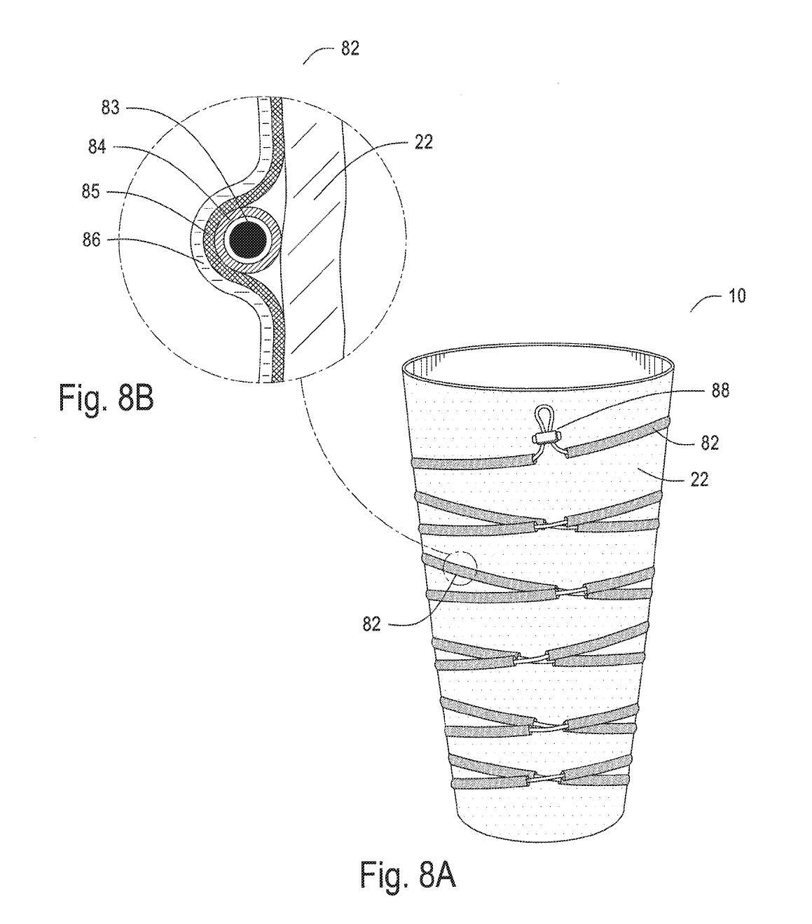

FIG. 8A is a side, perspective view of an embodiment of prosthetic socket liner garment 10, with a tensioning cable system 82 applied circumferentially around the external surface of garment 10. Cable system 82 may be tightened or loosened by one or more cable tension adjustment mechanisms, such as cable lock 88. FIG. 8B is a detailed, cross-sectional view of a segment of cable system 82, showing a cable 83 slidably encased within a sheath 84. Sheath 84 is covered by a thermoplastic exterior layer 86, with an intervening layer of thermoplastic hot melt adhesive 85 that secures cable system 82 to the exterior surface of prosthetic socket liner garment 10.

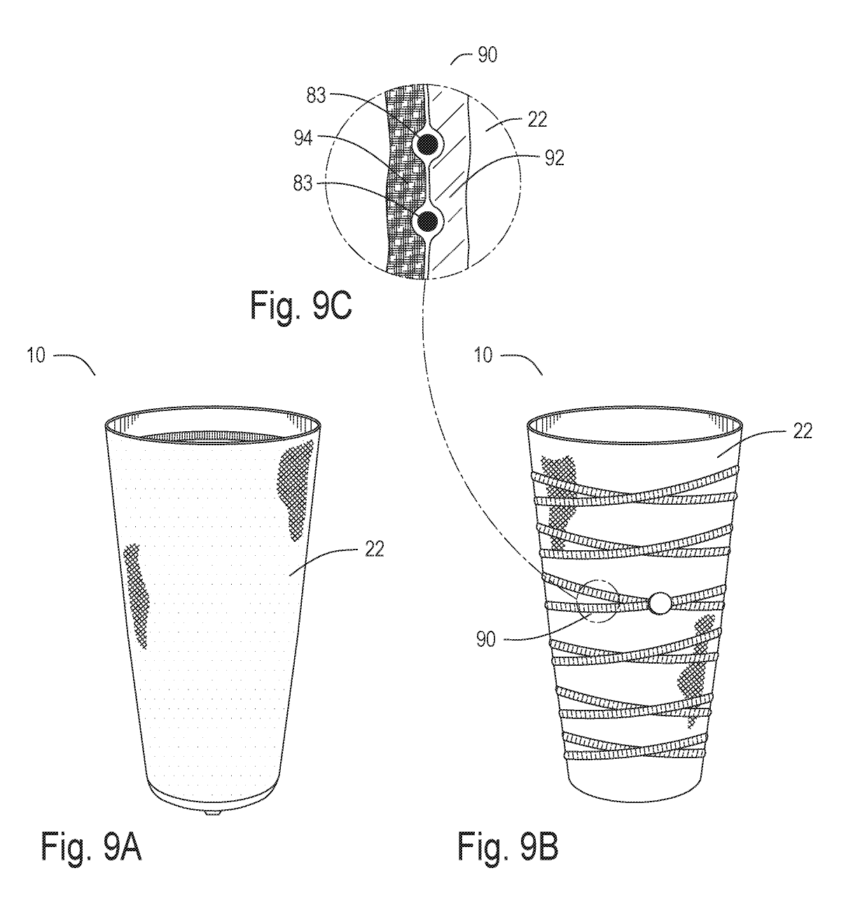

FIGS. 9A-9C show an alternative embodiment of a cable tensioning system 90, which is disposed within a woven fabric portion of prosthetic liner garment 10. FIG. 9A shows a side perspective view of liner garment 10, in which the cables are not visible, because they are disposed within a woven fabric portion of liner garment 10. FIG. 9B shows a view in which an external woven layer of liner garment 10 is removed, thus allowing a view of the underlying cable tensioning system 90. FIG. 9C shows a detailed, cross-sectional view of a portion of cable tensioning system 90, showing cables 83 securely disposed between an internal woven layer 92 and an external woven layer 94, both of which may be external to, integrated into or adhered to breathable fabric layer 22.

FIGS. 10A-10B show an embodiment of prosthetic socket liner garment 10, with a structural brace 31 integrated into the fabric of the garment. Brace 31 may include a relatively inelastic material 36, which is less elastic than layer 22, and may optionally include one or more tensioning members 37. FIG. 10A depicts prosthetic socket liner garment 10 in its entirety, with brace 31 in place. FIG. 10B shows brace 31 in isolation.

Referring now to FIGS. 11A-11P, prosthetic socket liner garment 10 may be secured within a prosthetic socket by a distal umbrella 40 and a distal funnel 60, which engage each other in a manner that precludes rotation of liner garment 10 with respect to distal funnel 60. Distal funnel 60 is securely disposed with a prosthetic socket (not shown); consequently the anti-rotational engagement of umbrella 40 and distal funnel 60 also precludes rotation of liner garment 10 with respect to a host prosthetic socket frame.

FIGS. 11A-11P are various views of one embodiment of distal umbrella 40 and distal funnel 60. FIGS. 11A-11B are top and bottom perspective views, respectively, of umbrella 40. Umbrella 40 has a top or proximal surface 42, a bottom or distal surface 44, and a central through hole 49, which is hexagonal in some embodiments. Radially aligned ribs 45 may be disposed across the bottom surface 44 of umbrella 40.

Referring to FIG. 11C, after a user has donned prosthetic socket liner garment 10, as the user inserts his or her residual limb into an awaiting prosthetic socket frame, distally projecting ribs 45 of umbrella 40 contact a proximal or top surface 62 of distal funnel 60. As umbrella 40 and distal funnel 60 come into closer apposition, ribs 45 of umbrella 40 seat into valleys between proximally projecting teeth 63 of distal funnel 60. By virtue of distal funnel 60 being securely and non-rotationally locked into the host prosthetic socket frame, so too is umbrella 40 secured in a manner that precludes rotation of liner garment 10 within the prosthetic socket frame.

FIG. 11C is a top, perspective view of a distal rotational locking funnel 60 for prosthetic socket liner garment 10. FIGS. 11D-11F are, respectively, a top perspective view, a side view, and a bottom view, of an umbrella 40 supported by a distal rotational locking funnel 60 for prosthetic socket liner garment 10.

FIGS. 11G-11K provide several types of side and cross-sectional views of umbrella 40 and distal funnel 60, to show aspects of the mechanism by which they can lock together. FIG. 11G shows a top perspective view of an umbrella 40 and distal rotational locking funnel 60 for a prosthetic socket liner garment 10, showing, in particular a reference plane that corresponds to the reference line in FIG. 11H. FIG. 11H shows a top face view of an umbrella 40 with a cross-sectional reference line pertinent to FIGS. 11I and 11J. FIG. 11I shows a detail side view of the engagement of distal ribs 45 of an umbrella 40 and proximal teeth 63 and intervening spaces of a distal rotational locking funnel 60 for a prosthetic socket liner garment. FIG. 11J shows a side face view of an umbrella 40 above a distal rotational locking funnel 60 with a reference line that corresponds to a cross-sectional slice as seen in FIG. 12K. FIG. 11K shows a cross-sectional view of an umbrella 40 above a distal rotational locking funnel 60, the cross-section corresponding to the reference line seen in FIG. 11J.

FIGS. 11L-11P provide several views of umbrella 40 and liner garment 10, to show aspects of how they may be bonded together. FIG. 11L is a side view of an embodiment of prosthetic socket liner garment 10, with pentagonal umbrella 40 bonded to its distal face. FIG. 11M shows a cross-sectional view of the same embodiment. FIG. 11N is a side perspective view of the same embodiment. FIG. 11O shows a side perspective view of the same embodiment, with pentagonal umbrella 40 spaced apart from its bonding site, showing seam lines 29 of darts 28 on the prosthetic socket liner garment 10. FIG. 11P shows a bottom face view of prosthetic socket liner garment 10 fitted with pentagonal umbrella 40.

As mentioned above, embodiments of the prosthetic liner garment system described herein generally include prosthetic liner garment 10, umbrella 40 bonded to the external distal aspect of liner garment 10, and distal funnel 60 engaging and securing umbrella 40 into a prosthetic socket frame. Various embodiments of prosthetic liner garment 10 and the other components of the prosthetic liner garment system may include any of a number of suitable materials, layers and combinations thereof, which are integrated into a unitary device, as shown particularly in FIGS. 1-3C. Liner garment 10 is typically an elongate, tube-like main body with an open proximal end, a closed distal end, and internal and external surfaces. The generally tubular structure may vary from being simply or substantially tubular, to being bulbous at the distal closed end, or to being conical, with a narrowing at the distal end. Embodiments of the prosthetic liner garment 10 may also be provided in various sizes and shapes in an inventory.

Prosthetic socket liner garment 10 may act: (1) to control an internal environment that hosts and supports a residual limb in a manner that is friendly to the residual limb; (2) to contribute to the suspension of the prosthetic socket, as a whole, on the residual limb; and (3) as an interface that contributes to the support and attachment of prosthetic components distal to the prosthetic socket. Distinct distal and proximal compartments within liner garment 10, and their roles in suspension of the prosthetic socket on the residual limb, will now be addressed. With respect to the patient and his or her residual limb, liner garment 10 embodiments provided herein may be appreciated as a compartment that hosts the residual limb. Embodiments of the liner garment 10 actually provide two compartments for the residual limb, a proximal compartment and a distal compartment, which are distinct from each other. These compartments correspond to the proximal sleeve portion 12 and the distal (gel) portion 14 of liner garment embodiments (FIG. 1). The distal compartment environment is substantially hermetically sealed, and by virtue of this seal, the distal portion of the liner garment 10 particularly contributes to suspension of a prosthetic device on the hosted residual limb. The distal compartment is also one where compression directed on the hosted residual limb by a liner garment 10 embodiment is substantially uniform throughout the compartment.

Embodiments of the invention may be sized and configured for residual limbs derived from amputations of either an upper or lower extremity, and further sized and configured for an amputation of any level within an upper extremity or a lower extremity (such as a transfemoral or transtibial amputation site). Dimensions, as cited below and elsewhere refer most particularly to a transfemoral amputation, but that is for exemplary purposes. Drawings herein generally depict an aspect ratio of width to length typical of a transfemoral amputation, but again, that is in reference to one example that does not exclude a residual upper extremity, or any amputation that leaves a residual limb of a different length would be suited for depicted liner garment examples. As emphasized elsewhere herein, a key aspect of novelty relates to a significant proximal portion of the full length of a liner garment 10 that is breathable.

In one particular embodiment, the distal portion 14 of a liner garment 10 has a length of about 140 mm (about 5.5 inches), the proximal portion has a length of about 260 mm (about 10.25 inches), and the full length of the garment 10 is about 400 mm (about 15.75 inches). Thus, in this particular example, the distal portion (with a silicon cup) accounts for about 35% of the total length of the liner garment 10, and the proximal (breathable portion) accounts for about 65% of the total length of the liner garment 10. In practical terms, a prosthetist can trim the full length of the liner garment 10 to fit a patient appropriately, so the proximal portion can vary in length independently of the distal portion. In related embodiments, the distal portion of the liner garment 10 may range in length between about 100 mm (3.9 inches) and about 175 mm (6.9 inches). Typically, liner garment 10 embodiments within an inventory of differently sized liner garments (FIGS. 6A-6C) will have a constant distal portion length, and the proximal portion length will vary from garment to garment, to fit the patient. At least one inventive aspect of liner garment 10 relates particularly to the fact that the distal portion of the liner, the portion comprising an encompassing gel layer, occupies but a fraction of the total length of the liner, whereas in typical prior art liners, the a gel layer occupies the full length of the liner.

In contrast to the distal portion, the proximal compartment 12 of the liner garment 10, bound by elastic fabric, is breathable, being permeable to air and fluid, and further providing a liquid wicking substrate. And although the compression and patterns of elasticity driving the compression can be uniform throughout the proximal compartment, such patterns may also be variable and customizable to be patient-specific. Breathability contributes to the health of the residual limb, specifically avoiding the unwanted effects of subjecting the residual limb to potential accumulation of heat and moisture. Variable compression and tensioning is controlled by varying the composition and structure of the fabric in specific, controllable, and customizable patterns of elasticity, inelasticity, and tensioning. The form of these patterns is designed to provide biomechanical advantages in general, as well as to accommodate personal preference considerations of the patient. Taken together, these patterns of elasticity and tensioning also contribute to the suspension of a prosthetic device on the patient's residual limb.

Accordingly, the proximal 12 and distal 14 compartments of the provided liner garment 10 embodiments each contribute to suspension of a prosthetic device. The use of hermetic sealing for suspension is important, but the minimization or containment of the hermetically sealed environment to the distal compartment is advantageous for the health of the residual limb.

Aspects of integrated layers and materials, and distribution thereof within liner garment 10 will now be addressed (FIGS. 3A-3C). The multiple materials of liner garment 10 embodiments may be distributed into multiple layers, typically two or more layers within the liner garment 10 as a whole, the multiple layers bonded, glued, annealed, woven, or otherwise made into a single integrated whole. The integrated layers included within the garment 10 may be distributed selectively in various ways, as for example: (a) a wholly uniform manner throughout the garment 10, (b) a uniform or non-uniform manner when comparing proximal and distal portions, (c) a uniform or non-uniform manner with respect to circumferential position or distribution, (d) a uniform or non-uniform manner with respect to cross-sectional distribution through a single thickness profile across all layers, and/or (d) a uniform or non-uniform manner with respect to regional or zoned locales within or across the surface area of the liner embodiment.

Some embodiments of the liner garment 10, as a whole, may include a gel layer 23 and a fabric layer 22 that has breathable, permeable, and wicking qualities (FIGS. 2A-3B). In terms of cross-sectional distribution, a gel layer, when present at a locale within a liner garment embodiment 10, is internal. A wicking layer may be either internal and/or external to the gel layer, and a fabric layer may be internal, external, or it may account for the totality of a locale.

With regard to surface area variation throughout a liner garment 10: a particular surface area sector may include variation within it, as for example, a particular region standing out as an island, peninsula, or strip, or more complex shape, against one or more neighboring dissimilar regions.

Further, there may be variation with regard to the total number of layers or materials present in a liner garment 10. For example, while the garment 10, as a whole, includes at least two types of layered material layers (a gel layer and a fabric layer), not all sites or regions of the garment 10 need to include these layers or a full complement of materials. By way of example, in particular embodiments of the garment 10, a contiguous or encompassing gel layer is included only in the distal portion 14 of the garment 10, to the exclusion of proximal portion 12 In another example, a wicking layer and a fabric layer may be effectively integrated into a single layer, or single heterogeneous fabric.

An external fabric layer 22, in particular, may take many forms and incorporate a number of special features. Embodiments of the fabric layer, or regions of the layer, may have a biased or have a 1-axis elastic property as a basic matrix, i.e., being elastic horizontally or circumferentially, but being substantially inelastic vertically. Some embodiments of the fabric layer, or regions of the layer, may have a 2-axis elasticity, i.e., being elastic both vertically and horizontally. In addition, the fabric may include regions of particular elastic strength or power, it may include regions that are substantially inelastic, it may incorporate regions of mesh or foam to serve as padding, it may include regions that are particularly adapted to wick fluid, and it may include regions re-enforced particularly for durability or strength.

Further, embodiments of the provided prosthetic socket liner garments may include other layers, physically distinct from the gel layer or fabric layer, that are dedicated to a particular functionality, such as a layer of padding (albeit, all layers being integrated into a functional whole), or a layer having a wicking substrate, or a layer that is particularly elastic, inelastic, or durable.

The tubular configuration of liner garment 10 originates as a flat pattern 10FP (FIG. 7). Embodiments of the prosthetic socket liner include a multilayer fabric portion that has a connecting vertical seam, and distal darted sections 28. Briefly, an embodiment of a prosthetic liner is fabricated as follows. Beginning as a flat piece 10FP (FIG. 7) cut from a pattern, two longitudinal sides are joined together so as to assume the tubular configuration of a liner, and to form a vertical seam 29. At the distal end of a liner, the darts 28 of the flat piece are joined together in seams to form a rounded distal end. In alternative embodiments, more than two or more flat pieces may be used, thereby creating more than one vertical seam. In a particular embodiment, the flat fabric pattern 10FP has five distal darted sections 28, although alternative embodiments may include fewer or more than five darted sections. A five-dart configuration may be considered advantageous because five darts, when joined, are sufficient to form an effectively rounded distal hemispherical profile of the liner garment 10, and five darts represent a minimal but sufficient number of darts to achieve such a satisfactorily rounded profile is advantageous for the simplicity of design.

Five darts 28, when joined together, create a five-pointed star arrangement at the distal end of a liner (FIGS. 6A-6C). This five-pointed star forms a site to which a pentagonal supportive umbrella 40 can be bonded, as described further elsewhere. The bonding of pentagonal umbrella 40 over the dart seams advantageously strengthens the seams 29. Certainly, some embodiments of flat fabric pattern 10FP may have fewer or more than five darts, in which case a companion supportive umbrella would advantageously have the same number of peripheral sides.

The distal gel layer of the prosthetic liner garment 10 may be formed separately and incorporated into the liner as a final step. In one example (FIG. 5), a silicone gel cup 30 mounted on a mandrel 32, covered with a layer of bonding resin, and inserted into the liner garment 10, and bonded thereto, onto the internal surface of the liner.

Aspects of liner garment 10 that relate to a gel layer in the form of silicone gel cup 30 within the distal portion of the liner will now be addressed (FIG. 1 and FIG. 3A). Although prior art liner garments typically have an internal and encompassing gel layer throughout their length, the present inventors have observed that such a full length gel layer is not necessary to provide effective suspension of a prosthetic socket from a liner garment, particularly when sufficient circumferential tensioning is applied to the proximal portion of the liner garment. Accordingly, in some embodiments of the provided liner garment, an internal gel layer is present only in the most distal portion of the liner. By way of example, a gel portion may have a length in the range of about 100 mm-about 170 mm of length.

The demarcation between the portions of the liner garment 10 that has (1) a gel layer and (2) the portion that does not have a gel layer may be considered (for purposes of this application) the demarcation between the distal portion 14 and proximal 12 portions of the liner garment, and also marks the boundary between two compartments from the perspective of the hosted residual limb. According, when being worn, the distal portion 14 of the liner garment 10 establishes a substantially sealed portion or compartment with respect to the residual limb, the gel layer being adherent or substantially adherent against the skin of the residual limb. The adherent gel layer hermetically seals the compartment in the distal portion of the liner garment, preventing influx or efflux of air in that distal space. A combination of gel-skin adherence and external air pressure favors stabilization of liner garment 10 on the residual limb.

In some embodiments of liner garment 10, the proximal portion 12 of prosthetic socket liner embodiments may also include silicone gel sections 23 on the internal surface of the liner garment arranged in various patterns. However, typically, these gel patterns do not form a continuous circumferential arrangement. These gel patterns can contribute to the stability or adherence of the proximal portion of the liner on the residual limb without creating a hermetic environment such as that within the distal portion of the liner.

Aspects of liner garment 10 that relate to a wicking substrate will now be addressed. In typical embodiments of liner garment 10, a fabric or fabric-foam-fabric layer 22 is contiguous throughout the prosthetic socket liner garment, with other layers or segments adhered either side of such layer, internally or internally. Such a fabric or fabric-foam-fabric layer 22 is breathable, allowing passage of air and moisture, moisture movement occurring either through bulk liquid flow, by wicking, or by transpiration. In general, movement of air or moisture to the external environment occurs only in the proximal portion of the liner garment 10 because the distal portion of the garment includes an internally disposed silicone gel layer that is fluid impermeable. Inasmuch as the proximal portion 12 of liner garment 10 embodiments is breathable, it does not contribute to suspension of the liner on the residual limb by way of a forming a hermetic seal that resists movement against or away the skin as does the distal portion 14 of the garment; however, in some embodiments, the distal portion of the liner garment 10 contributes to suspension of the liner garment through the action of internally or externally disposed tensioning elements incorporated into or adherent to the fabric layer.

In some embodiments, a wicking substrate may further be included in the distal portion of the garment 10 as well, albeit distributed in such a manner so as not to disrupt the hermetic seal. Aspects of moisture management by way of wicking substrates included within a liner garment are described in U.S. patent application Ser. No. 14/310,147 (U.S. Patent Publication No. 2014/0379097) of Hurley and Williams, which is incorporated herein by this reference.

Aspects of liner garment 10 that relate to a configurations and tensioning of a proximal sleeve portion 12 of liner garment embodiments (FIGS. 4A-4C, 8A-8B, 9A-9C, and 10A-10B) will now be addressed. Embodiments of the proximal or sleeve portion 12 of the liner garment 10 may assume a number of configurations by virtue of variations in the composition of the fabric portion. In typical embodiments, the fabric has a pattern of biased elasticity; i.e., it is elastic to varying degrees circumferentially or along a horizontal axis, but substantially inelastic longitudinally, along a vertical axis.

In addition to this biased elasticity feature as a baseline, other features may be integrated into- or adhered to the fabric in a manner that modifies overall elasticity and circumferential tensioning. Embodiments of the liner garment 10 may include two basic classes of tensioning mechanisms: (1) fabric-based tensioning members 37 and (2) cable-based tensioning elements (82, 90).

As an example of fabric-based tensioning members 37 (FIGS. 10A and 10B), substantially inelastic strips may be integrated into the fabric, or applied to the fabric internally or externally. These inelastic strips are typically oriented longitudinally, but can vary from that generality and form various patterns that include diagonals, joined sections, islands, peninsulas, and biomechanically favorable patterns. Regions of the sleeve or proximal portion 12 may have such substantially inelastic tensioning members 37, arranged deliberately and rationally to create desired functional patterns of elasticity and inelasticity.

Similarly, tensioning elements or regions that create an internally- or centripetally directed compression on the residual limb may be may be integrated into the fabric, or applied internally or externally. External tensioning elements may include separate sections that can be joined, thereby adding an adjustable aspect to the tensioning. Tensioning elements are typically oriented circumferentially, but can vary from that generality and can form various patterns that include diagonals, joined sections, islands, and peninsulas. Regions of the sleeve or proximal portion 12 of a liner garment 10 may have such substantially elastic features arranged deliberately and with a biomechanical rationale to create desired functional patterns of higher and lesser degrees of circumferential compression. In one example, a combination of relatively inelastic region and tensioning regions may assume the form of a biomechanically appropriate brace.

Aspect of the technology that related to cable-based tensioning mechanisms for liner garment 10 will now be addressed (FIGS. 10A-10B). As noted above, in addition to fabric-integrated tensioning elements that are relatively large and apply tension to relatively large expanses of liner garment 10 surface area, embodiments of a liner garment may also include an embodiment of a cable-based tensioning system (82, 90). Cable-based tensioning embodiments, compared to the fabric based tensioning members 37 are more localized, but include a total length that magnifies local effect into a circumferential and broad surface effect.

Two types of cable-based tensioning systems are provided (FIGS. 8A-9B). In one embodiment 82, cables 83 are enclosed in a protective sheath 84 that is bonded to the external surface of a prosthetic liner garment. In one such embodiment (FIGS. 8A-8B), a lengthy cable is enclosed within a plastic sheath. Cable embodiments may be formed from any suitable material that maintains integrity under tension and shows no substantial distortion or stretch under tensioning forces encountered during uses such as those described herein, and can be effectively grasped by a Boa or cable lock mechanism. Examples of cable composition include stainless steel bundled wire, nylon or polyester lacing, or any suitable functional equivalent.

The sheath-enclosed cable 83 may be secured on the external surface of the liner garment 10 by a layer of thermoplastic urethane hot melt adhesive, and then covered with another layer of a durable thermoplastic urethane. This type of fabrication method may preclude the need for sewing of a plastic tubing or webbing on the fabric surface to secure the cable. The cable, freely slidable within its protective sheath, is generally wrapped around the liner garment 10 in a continuous way, although in some embodiments, more than one cable may be so arranged. The cable(s) may be tensioned by any suitable mechanism, such as a Boa connector or a cord-locking device.

In a second cable-based tensioning embodiment 90, tensioning cables 83 may be arranged in channels disposed within a fabric layer or between fabric layers of a liner garment (FIGS. 9A-9C). As with the external sheathed cable embodiment 82 described above, the cable is generally wrapped around the liner garment 10 in a continuous manner, although in some embodiments, more than one cable may be so arranged. Fabric of this type may be knit as a single piece on a flat knitting machine. The cable(s) may be tensioned by any suitable mechanism, such as a Boa connector or a cord-locking device.

By either approach described above (embodiments 82 and 90), each small segment of a tensioning cable exerts a highly localized compressive effect on the residual limb hosted within liner garment 10. However, considering the total length of a tensioning cable, the sum total of localized compressive effects integrates into a circumferential compressive effect. Further, the arrangement of the cable path can create regions of relatively low and relatively high compression. In particular embodiments, the path of a cable can be customized to a particular patient, thus creating regions of variable compression within the liner garment 10 that manifest as regional effects on the hosted residual limb.

Embodiments of a liner garment 10, as provided herein, include particular and distinct regions or zones within the surface area of the garment 10 that manifest as particular regional or zonal effects on a residual limb hosted within. By way of example, proximal 12 and distal 14 regions of liner garment 10 embodiments are distinct (FIG. 1). Proximal region 12 is breathable as a whole, while the distal region 14 is substantially impermeable to fluid. The breathable character of the proximal region contrasts with prior art liner garments that are substantially impermeable to fluid throughout their length. Breathability is advantageous in that fluid buildup within the liner can be substantially eliminated as a problem.

In a second example, the proximal portion of the liner garment 10 includes regions that are substantially inelastic and regions that are elastic. Elasticity is generally biased in that elasticity is allowed in a horizontal or circumferential dimension, but substantially disallowed vertically; these features are imparted by the nature of the weave of the fabric. In general, such biased elasticity is the default, baseline, or dominant character of the proximal portion of liner garment 10 embodiments; inelasticity is added to- or integrated within the dominant character of the fabric weave. Inelastic portions of the proximal portion of liner garment 10 embodiments may be appreciated as structural elements of liner that manifest as sites of compression on a hosted residual limb. Elastic portions, in contrast, provide a relative degree of relief from compression.

Embodiments of the prosthetic socket liner, as provided herein, may be fabricated in a matrix or inventory of varied sizes and shapes (FIGS. 6A-6C), and are customizable. Sizing may refer to both length and width (or circumference) of liner garment 10 embodiments. Shapes may include conical, tubular, or bulbous. With such variables, most patients can be well fitted with a prosthetic socket liner embodiment from an inventory of such sizes and shapes. Ancillary features included in a larger prosthetic system, such as a supportive umbrella 40 and a distal funnel 60 (described below) may also be provided in a matrix or inventory of varied sizes to complement the sizing of the prosthetic socket liner.

With regard to the various patterns of tensioning and elasticity within the sleeve portion 12 of liner garment 10 embodiments, in some embodiments these patterns are standardized according to functional generalities that apply to statistically common or consensus residual limbs. However, residual limbs are highly individual, and may differ from a consensus residual limb sufficiently that an individually customized arrangement of tensioning and elasticity has significant therapeutic benefit; accordingly, embodiments of the technology include the fabrication of customized arrangements of tensioning and elasticity.

Digital profiles of residual limbs can be captured by methods described in U.S. patent application Ser. No. 14/731,163 (US Patent Pub. No. 2015/0352775) of Geschlider et al., as filed on Jun. 4, 2015, which is incorporated herein by this reference. Digital profiles such as these can be directed to determine optimal arrangements of tensioning and elasticity. These profiles are typically directed toward a highly conformal model of the residual limb, but they may be further modified by biomechanical considerations, lifestyle or activity considerations, or personal preferences of the user. By such methods, highly customized arrangements (custom-fitted for an individual patient) of tensioning and elasticity can be created in liner garment 10 embodiments, particularly in the proximal sleeve portion. Custom sizing and regional arrangements of tensioning and elasticity are typically applied to fabrication of a prosthetic socket liner during the flat pattern stage of its fabrication, as described above.

Aspects of the provided technology that relate to a distal umbrella 40 (including a pentagonal periphery and radiating ribs 45 on the umbrella's distal surface) will now be addressed (FIGS. 11A-11P). Describing features of a distal supportive umbrella 40 can begin with an appreciation of some aspects of the distal end of a prosthetic socket liner. As noted above in the context of the five-pointed star formed by joined darts 28 at the distal end of a liner embodiment, an embodiment of umbrella 40 may be bonded thereto. A distally-positioned umbrella 40 provides a supportive and supportable structure onto the generally compliant form of a prosthetic socket liner. Embodiments of umbrella 40 have a generally concave form, i.e., a concavity facing proximally when bonded to the convex distal end of a socket liner. Embodiments of the umbrella 40 may be provided in various sizes in order to match the diameters of prosthetic socket liner sizes. In one example, embodiments of an umbrella 40 are formed by injection molding of Hytrel 7246 Black, or a functional equivalent thereof.

Embodiments of a distal umbrella 40 are generally saucer-shaped (FIG. 11A), concave on the proximal surface 42 and convex on the distal surface 44. The concavity of the proximal surface conforms to the convex distal aspect of a prosthetic socket liner garment 10. In accordance with a five-dart arrangement of the distal end of a prosthetic liner (described above), some particular embodiments of a supportive umbrella 40 are pentagonal. The points or peaks of the pentagonal shape correspond to the seams 29 that join the darts 28 of the prosthetic socket liner, and to overlay these seams when the umbrella 40 is bonded to the distal surface of the liner. As noted above in description of the seamed darts, the bonding of the umbrella 40 over the seams supports the integrity of the seams.

The distal surface of the supportive umbrella 40 includes a set of raised ribs 45 that radiate from a central hole toward the periphery of the umbrella (FIG. 11B). In a typical arrangement, these radiating ribs correspond in placement with the overall shape of the umbrella. In a pentagonal umbrella 40 embodiment, for example, a rib extends from the central hole toward each of the pentagonal points. One or more ribs may be positioned between this basic set of five ribs. Thus, in one example, a supportive umbrella 40 may have a total of ten ribs, five that are directed toward a pentagonal point, and five ribs, each disposed between two of the point-associated ribs. In an alternative view, the radiating ribs are fully diagonal, albeit interrupted by a central hole. In this view, one radial section of a rib is directed toward a pentagonal point, and the opposite radial section is directed toward a peripheral aspect of the umbrella 40 that is at midpoint between two neighboring pentagonal peaks.

These radiating ribs 45 provide at least two functions. In one aspect, they stiffen and strengthen the saucer shaped umbrella, and allow for it to be thinner than it would need to be without such ribs. In a second aspect, these radiating ribs are configured to engage with a set of teeth on the proximal surface of a distal funnel, as described further below. Ribs of the umbrella 40 and teeth of the distal funnel, together, form an anti-rotational locking mechanism. When the distally facing ribs of the umbrella 40 and the proximally facing teeth and intervening slots of the distal funnel engage, the umbrella 40 (and hence, liner garment 10 as a whole) cannot rotate within the prosthetic socket frame.

Some dimensional features of the provided umbrella 40 are particularly advantageous. Compared to prior art umbrella-like devices, the provided embodiment is notably wide; it has a circumference (particularly as defined by the pentagonal peaks) that is nominally equal to the circumference of the distal end of the liner to which it is bonded. Prosthetic socket liners, prior art liners in particular, can be problematic in that they can allow pistoning and/or rotation of the residual limb within a prosthetic socket, or within the confines of the prosthetic socket liner. Pistoning can allow a milking behavior, whereby a side of the liner, or some circumferential aspect of the liner collapses repeatedly during a patient's gait. Rotation and milking are irritating and potentially injurious to a residual limb, and thus highly undesirable. The circumference (or width) of the umbrella 40 provides a broad and substantially uniform support across its surface for a liner garment 10 to which it is bonded. The combination of wide and uniform support provided by umbrella 40 embodiments to the prosthetic socket liner disposed proximally to it discourages pistoning of a residual limb within the prosthetic socket liner.

In spite of its width (or circumference), embodiments of the umbrella 40 still permit an easy and effective reflection or eversion of the liner that is required when a patient is donning the liner. The pentagonal peaks account for the widest diameter, but the valleys between the peaks create a forgiveness or slack that allows the gel cup 30 portion to roll over the umbrella 40 as a whole. Another aspect of umbrella 40 includes a locking interaction of umbrella 40 with an underlying distal funnel 60 (as described below) precludes rotation of the liner within a hosting prosthetic socket.

Embodiments of a prosthetic socket liner technology may include various hardware elements, such as a distal funnel 60 configured to be disposed in the distal-most aspect of a prosthetic socket cavity (FIG. 11C). Embodiments of the funnel 60 may serve multiple purposes; for example, it may support an umbrella feature of a prosthetic socket liner, and it may provide a site of distal attachment for positioning slings within the prosthetic socket frame (as described in U.S. Provisional Patent Application No. 62/221,996 of Hurley et al., as filed on Sep. 22, 2015, and U.S. Provisional Patent Application 62/237,204 of Hurley et al., as filed on Oct. 5, 2015, U.S. Provisional Patent Application No. 62/287,702 of Hurley et al., as filed on Jan. 27, 2016. The present application, however, is directed toward the functionality of the distal funnel 60 in supporting an umbrella feature 40 of a prosthetic socket liner and acting as an anti-rotational mechanism. In one example, embodiments of a distal funnel are formed by machining of Black Delrin or a functional equivalent thereof.

Embodiments of distal funnel 60 are generally saucer-shaped; the proximal aspect 62 includes a broad central well with a hole 69 in the center (FIGS. 11D-11J). The raised periphery includes a set of proximally extending teeth 63 and intervening slots that are sized and configured to accommodate the distally extending ribs of the umbrella, as described above. When distal funnel 60 is installed in the cavity of a prosthetic socket, it is bolted in. When a patient is donning a prosthetic socket liner embodiment, as provided herein, the distally projecting ribs contact the proximal surface of the distal funnel and settle in to the slots between the proximally-extending funnel teeth. Inasmuch as the funnel is secured to the prosthetic socket frame and cannot rotate, so too is the umbrella 40 portion of the prosthetic liner then rotationally locked in place. The prosthetic socket liner, by way of the umbrella and funnel is vertically secured within the socket by any of a lanyard, a locking pin arrangement, or a vacuum based arrangement.

Any one or more features of any embodiment of the inventions disclosed herein (device or method) can be combined with any one or more other features of any other embodiment of the inventions, without departing from the scope of the invention. It should also be understood that the invention is not limited to the embodiments that are described or depicted herein for purposes of exemplification, but is to be defined only by a fair reading of claims appended to the patent application, including the full range of equivalency to which each element thereof is entitled.

What is claimed is:

1. A liner garment for a prosthetic socket configured to accommodate a residual limb of a patient, the liner garment comprising:
a main body, comprising an elastic, fluid permeable material layer extending around a full circumference of the liner garment and having an inner surface, an outer surface, a closed distal end and an open proximal end for accepting the residual limb of the patient;
a fluid impermeable gel cup positioned on the inner surface of the main body at its distal end, wherein the gel cup is configured to accommodate a distal end of the residual limb and has a total gel cup length of at least 100 millimeters; and
at least one support material layer on a portion of the outer surface of the main body, wherein the at least one support material layer comprises a material that is less elastic than the fluid permeable material layer of the main body.

2. The liner garment of claim 1, wherein the at least one support material layer is attached to the outer surface of the main body by a method selected from the group consisting of bonding, gluing and interweaving.

3. The liner garment of claim 1, wherein the at least one support material layer comprises a pattern of multiple semi-elastic strips disposed around the circumference of the liner garment, while leaving some portions of the main body outer surface uncovered by the support material layer.

4. The liner garment of claim 3, further comprising multiple inelastic strips disposed on outer surfaces of at least some of the semi-elastic strips.

5. The liner garment of claim 4, further comprising a silicone gel layer disposed in a pattern on the inner surface of the main body, while leaving some portions of the main body inner surface uncovered by silicone gel layer.

6. The liner garment of claim 5, wherein the silicone gel layer comprises multiple strips of silicone gel material.

7. The liner garment of claim 1, wherein a proximal portion of the main body and the gel cup form two internal compartments within an interior of the liner garment, the two compartments comprising:
a breathable proximal internal compartment; and
a distal hermetically sealed internal compartment.

8. The liner garment of claim 1, wherein the main body comprises a layer of foam disposed between two layers of fabric.

9. The liner garment of claim 8, wherein the foam layer comprises neoprene.

10. The liner garment of claim 1, wherein the gel cup comprises a material selected from the group consisting of silicone gel, urethane gel and thermoplastic elastomer gel.

11. The liner garment of claim 1, wherein the gel cup is attached to the inner surface of the main body at its distal end and does not extend all the way to the proximal end of the main body.

12. The liner garment of claim 11, wherein the total gel cup length is no greater than 170 millimeters.

13. The liner garment of claim 1, wherein the main body comprises a woven fabric having a biased elasticity that allows a proximal portion of the main body to stretch circumferentially not vertically.

14. The liner garment of claim 1, wherein the at least one support material layer comprises a pattern of polyurethane laminated on the outer surface of the main body.

15. The liner garment of claim 1, wherein the at least one support material layer comprises a pattern of polyurethane integrated into the main body such that it is exposed on the outer surface.

16. The liner garment of claim 1, wherein the at least one support material layer comprises non-stretch webbing applied to the outer surface of the main body.

17. The liner garment of claim 1, further comprising at least one additional support material layer applied to the inner surface of the main body.

18. The liner garment of claim 17, wherein the at least one additional support material layer comprises non-stretch webbing applied to the internal surface.

19. The liner garment of claim 1, wherein at least one support material layer comprises a brace member disposed over part of the outer surface of the main body.

20. The liner garment of claim 19, wherein the brace member comprises at least one tensioning member.

21. The liner garment of claim 1, further comprising a hard, supportive umbrella attached to the outer surface of the distal end of the main body.

22. The liner garment of claim 21, wherein the umbrella has a concave proximal surface that conforms to the outer surface of the distal end of the main body.

23. The liner garment of claim 22, wherein the umbrella has a pentagonal shape, and wherein five points of the pentagonal shape are disposed over five centrally converging seams of the distal end of the main body.

24. The liner garment of claim 21, wherein the umbrella comprises:
a distal surface; and
multiple radiating ribs disposed on the distal surface, wherein the ribs are configured to engage a complementary set of slots disposed on a proximal surface of a distal funnel on a distal-most aspect of a cavity of a prosthetic socket.

25. The liner garment of claim 21, wherein the umbrella comprises a contact surface in contact with the distal end of the main body, and wherein the contact surface has an area of between 15.9 cm² and 29.2 cm².

26. The liner garment of claim 21, further comprising a distal funnel configured for placement at a distal-most end of a cavity of the prosthetic socket.

27. The liner garment of claim 26, wherein the distal funnel comprises a proximal surface, having a set of teeth and intervening slots configured to complement a shape of radiating ribs disposed on a distal surface of the umbrella.

28. The liner garment of claim 1, further comprising a cable-based tensioning mechanism disposed over at least part of the outer surface of the main body.

29. The liner garment of claim 28, wherein a cable of the cable-based tensioning mechanism is enclosed in a plastic sheath adhered to the outer surface of the main body by one or more layers of thermoplastic.

30. The liner garment of claim 28, wherein the cable-based tensioning mechanism is disposed within a fabric layer of the main body and arranged in multiple circumferential loops around the main body.

31. A liner garment system for a prosthetic socket configured to accommodate a residual limb of a patient, the system comprising:
a main body, comprising an elastic, fluid permeable material layer extending around a full circumference of the liner garment and having an inner surface, an outer surface, a closed distal end and an open proximal end for accepting the residual limb of the patient;
a fluid impermeable gel cup positioned on the inner surface of the main body at its distal end, wherein the gel cup is configured to accommodate a distal end of the residual limb and does not extend to the proximal end of the main body;
at least one support material layer on a portion of the outer surface of the main body, wherein the at least one support material layer comprises a material that is less elastic than the fluid permeable material layer of the main body;
a supportive umbrella attached to the outer surface of the distal end of the main body; and
a distal funnel configured for placement at a distal-most end of a cavity of the prosthetic socket, wherein the distal funnel comprises a proximal surface that is complementary in shape to a distal surface of the umbrella.

32. The system of claim 31, wherein the at least one support material layer comprises a pattern of multiple semi-elastic strips disposed around the circumference of the liner garment.

33. The system of claim 32, further comprising multiple inelastic strips disposed on outer surfaces of at least some of the semi-elastic strips.

34. The system of claim 31, further comprising a silicone gel layer disposed in a pattern on the inner surface of the main body.

35. The system of claim 31, wherein the main body comprises a layer of neoprene disposed between two layers of fabric.

36. The system of claim 31, wherein the gel cup comprises a material selected from the group consisting of silicone gel, urethane gel and thermoplastic elastomer gel.

37. The system of claim 31, wherein the gel cup has a length of no greater than 170 millimeters.

38. The system of claim 31, wherein the at least one support material layer comprises polyurethane.

39. The system of claim 31, wherein the umbrella has a concave proximal surface that conforms to the outer surface of the distal end of the main body.

40. The system of claim 39, wherein the umbrella further comprises:
a distal surface; and
multiple radiating ribs disposed on the distal surface, wherein the ribs are configured to engage a complementary set of slots disposed on a proximal surface of the distal funnel.

41. The system of claim 40, wherein the proximal surface of the distal funnel further comprises a set of teeth disposed between the slots, and wherein the ribs of the umbrella and the slots and teeth of the distal funnel fit together to lock the umbrella to the distal funnel to prevent rotation of the umbrella relative to the distal funnel.

42. The system of claim 31, further comprising a cable-based tensioning mechanism disposed over at least part of the outer surface of the main body.
